(19) United States
(12) Patent Application Publication
(10) Pub. No.: US 2024/0285566 A1
(43) Pub. Date: Aug. 29, 2024

(54) SUBCUTANEOUS FORMULATION AND METHODS FOR TREATING EDEMA REFRACTORY TO ORAL DIURETICS

(71) Applicant: RESQ Pharmaceuticals LLC, Orange, CA (US)

(72) Inventors: Balasingam RADHAKRISHNAN, Chapel Hill, NC (US); Ben ESQUE, Huntington Beach, CA (US); Wei LIN, San Diego, CA (US); Andrew Xian CHEN, San Diego, CA (US)

(21) Appl. No.: 18/568,440

(22) PCT Filed: Jun. 9, 2022
(86) PCT No.: PCT/US2022/032862

§ 371 Date: Dec. 8, 2023

Related U.S. Application Data
(60) Provisional application No. 63/208,863, filed on Jun. 9, 2021.

(51) Int. Cl. A61K 31/196; A61K 9/00; A61K 9/08; A61K 47/10; A61K 47/26

(52) U.S. Cl. CPC A61K 31/196; A61K 9/0019; A61K 9/08; A61K 47/10; A61K 47/26

(57) ABSTRACT

The present invention features methods and compositions for the subcutaneous administration of bumetanide for the treatment of subjects suffering from edema refractory to oral diuretics.

### FIELD OF THE INVENTION

The disclosure features methods and compositions for subcutaneous injection for the treatment of edema refractory to oral diuretics.

### BACKGROUND OF THE INVENTION

Congestive heart failure (CHF) is a common heart disease. The prevalence of incidents of congestive heart failure has recently increased, and there is considerable morbidity and mortality-associated with its diagnosis. In fact, congestive heart failure is an extremely lethal disease with an estimated five-year mortality for a vast majority of both men and women who encounter the disease. Congestive heart failure results from loss of, or impairment of, normal heart function. This loss or impairment reduces cardiac output. This, in turn, results in a reduction in both blood flow and blood pressure in the kidneys. This reduction in flow and pressure causes a renin-angiotensin response that exacerbates congestive heart failure. Blood volume is increased because angiotensin II stimulates secretion of aldosterone from the adrenal cortex which, in turn, causes an increase in salt and water retention in the kidneys. The increase in blood volume and the corresponding vasoconstriction cause an increase in blood pressure and hence fluid overload on the heart which causes further deterioration of the heart condition.

To treat CHF, physicians put patients on a strict low sodium diet and their fluid intake is monitored. Some patients are limited to as little as one liter of fluid a day. The most important drugs in the physician's arsenal to combat fluid overload are the class of drugs called diuretics. Diuretics affect the kidney function in such a way that the reabsorption of fluid is suppressed. As a result, there is more urine output contrary to neurohormonal commands that the kidney is receiving. Physicians can treat the patient with agents that improve the pumping ability of the heart, increase blood pressure, and attempt to reactivate a more normal behavior of the body's control system. In general, this is effective in sustaining life of many heart failure patients. Nevertheless, in hundreds of thousands of patients, treatments with drugs and diet alone fail. When the patient is in an edematous state and is experiencing fluid overload, gastrointestinal absorption can be compromised, thus limiting the effectiveness of oral diuretics. As a result, the patient is often instructed to increase the dosage of oral diuretics putting further strain on the patient's kidneys. Eventually, oral diuretics become insufficient to remove excess fluid causing the patients to seek intravenous diuretics to bypass absorption by the gastrointestinal system. Consequently, the patients are often repeatedly admitted to the hospital for intensive care and administration of iv diuretics and are at risk (with each event) of over-diuresis once gastrointestinal absorption is restored. Ultimately, the over-diuresis can result in kidney failure. When available treatment can no longer achieve adequate fluid removal with existing kidney function, renal replacement therapies such as hemofiltration or dialysis have been increasingly used as a method of removing fluid in the acute CHF state. Acute heart failure can be treated with the Continuous Renal Replacement Therapy (a.k.a., an artificial kidney or dialysis machine) in the ICU of a hospital.

Therefore, there remains a need for treatment options for patients with heart failure in acute distress from fluid overload, such as to reduce the risk of hospitalizations and kidney failure.

### SUMMARY OF THE INVENTION

The invention features subcutaneous compositions and methods for the treatment of edema refractory to oral diuretics.

In a first aspect, the invention features a method of treating edema in a subject, the method including administering subcutaneously an effective amount of a pharmaceutical composition including (i) an aqueous solution having a pH of between about 5 and about 9 (e.g., a pH of 5±1, 6±1, 7±1, 8±1, or 9±1), (ii) between about 4 mg/mL and about 20 mg/ml (e.g., 4±1, 5±1, 6±1, 7±1, 8±1, 9±1, 10±1, 11±1, 12±1, 13±1, 14±1, 15±1, 16±1, 17±1, 18±1, 19±1, or 20±1 mg/mL) arginine bumetanide salt, and (iii) one or more pharmaceutically acceptable excipients. In particular embodiments, the pharmaceutical composition includes (i) an aqueous solution having between about 5 mg/mL and about 12 mg/mL arginine bumetanide salt, (ii) one or more pharmaceutically acceptable excipients, and (iii) wherein the aqueous solution has a pH of between about 6 and about 8.

In some embodiments, the pharmaceutically acceptable excipients include a tonicity agent (e.g., any tonicity agent described herein) or a buffering agent (e.g., citrate buffer, phosphate buffer, or any other buffering agent described herein). In particular embodiments the aqueous solution is free of a buffering agent other than the buffer formed by bumetanide free acid combined with arginine.

In a first aspect, the invention features a method of treating edema in a subject, the method including administering subcutaneously an effective amount of a pharmaceutical composition including (i) an aqueous solution having a pH of between about 5 and about 9 (e.g., a pH of 5±1, 6±1, 7±1, 8±1, or 9±1), (ii) between about 4 mg/ml and about 20 mg/ml (e.g., 4±1, 5±1, 6±1, 7±1, 8±1, 9±1, 10±1, 11±1, 12±1, 13±1, 14±1, 15±1, 16±1, 17±1, 18±1, 19±1, or 20±1 mg/mL) potassium bumetanide salt, and (iii) one or more pharmaceutically acceptable excipients. In particular embodiments, the pharmaceutical composition includes (i) an aqueous solution having between about 5 mg/mL and about 12 mg/mL potassium bumetanide salt, (ii) one or more pharmaceutically acceptable excipients, and (iii) wherein the aqueous solution has a pH of between about 6 and about 8.

In some embodiments, the pharmaceutically acceptable excipients include a tonicity agent (e.g., any tonicity agent described herein) or a buffering agent (e.g., citrate buffer, phosphate buffer, or any other buffering agent described herein). In particular embodiments the aqueous solution is free of a buffering agent other than the buffer formed by bumetanide free acid combined with potassium hydroxide.

In some embodiments, the aqueous solution has a pH of between about 6 and about 8. In some embodiments, the one or more pharmaceutically acceptable excipients includes a sugar or sugar alcohol (e.g., sucrose and/or mannitol). In some embodiments, the pharmaceutical acceptable excipient includes a preservative agent (e.g., benzyl alcohol).

The invention further features a method of treating edema in a subject in need thereof, the method including administering to the subject an effective amount of a pharmaceutical composition of the invention. In some embodiments, the pharmaceutical composition is administered in a dose volume of no more than 0.5 mL The administering can include delivering a dose volume of from 25 μl to 500 μl (e.g., 35±10, 50±10, 75±25, 125±25, 150±25, 200±50 μl, 300±100 μl, or 400±100 μl) of the pharmaceutical composition subcutaneously to the subject. In particular embodiments, the dose is delivered not more than from 1 to 4 times over a six hour period. In certain embodiments, four doses of about 400 μL each are delivered over a period of 1 hour. In particular embodiments, two doses of about 400 μL each are delivered to a subject followed by two more does of about 400 μL each after a period of about 30 to 60 minutes. In other embodiments, the subject is suffering from edema refractory to oral diuretics. In certain embodiments, the subject has congestive heart failure. In particular embodiments, the subject is suffering from edema in the lung (e.g., pulmonary edema). In some embodiments, the pharmaceutical composition is administered subcutaneously to the subject in an outpatient setting. In certain embodiments, the pharmaceutical composition is self-administered.

In an embodiment of any of the above methods, the pharmaceutical composition has a bioavailability of at least 90% (e.g., 91%, 92%, 93%, 94%, 95%, 96%, 97%, 98%, 99%, or 100%) or 95% (e.g., 96%, 97%, 98%, 99%, or 100%) upon subcutaneous administration to the subject.

In particular embodiments, the pharmaceutical composition includes a buffer selected from citrate buffer and phosphate buffer.

In some embodiments, the pharmaceutical composition is administered subcutaneously to the subject in an outpatient setting or the pharmaceutical composition is self-administered. In certain embodiments, the subject is experiencing reduced intestinal motility prior to the administering.

The method can be of particular benefit where the subject has failed to achieve diuresis with oral diuretic therapy prior to the subcutaneously administering. The subject to be treated may be experiencing swelling of the legs, shortness of breath, difficulty breathing, or chest pain unresolved with oral diuretic therapy prior to the subcutaneously administering. In some embodiments, the subject is experiencing reduced intestinal motility prior to the subcutaneously administering.

In a related aspect, the invention a method of treating edema refractory to oral diuretics in a subject with congestive heart failure, the method including administering to the subject an effective amount of a pharmaceutical composition including any one of the pharmaceutical compositions described herein.

In an embodiment of any of the above methods, the subject has been treated with at least one dosage of an oral diuretic within the last 24 hours prior to the administering. The at least one oral diuretic can be selected from loop diuretics, such as bumetanide, furosemide or torsemide, or potassium-sparing diuretics, such as amiloride or spironolactone.

In some embodiments of any of the above methods, the patient does not receive more than a total of about 10 mg of the bumetanide salt over a 12 hour period. In some embodiments, the patient consults a physician having administered more than a total of 10 mg of the bumetanide salt over a 12 hour period.

In some embodiments of any of the above methods, the subject's risk of hospitalization due to complications associated with edema is reduced.

In an embodiment of any of the above methods, the pharmaceutical composition is administered in one, two, three or four doses over a 12 hour period. In some embodiments, the pharmaceutical composition is administered in a single dose. In some embodiments, the dosage over a 12 hour period from 1 to 2.5 mg of the bumetanide salt is administered in one, two, three, or four doses. In some embodiments, the dosage over a 12 hour period is from 2 to 5 mg of the bumetanide salt is administered in one, two, three or four doses. In some embodiments, the dosage over a 12 hour period is from 3 to 7.5 mg of bumetanide salt is administered in one, two, three or four doses. In some embodiments, the dosage over a 12 hour period is from 4 to 10 mg of bumetanide salt is administered in one, two, three or four doses.

In a related aspect, the invention features a pharmaceutical composition including (i) an aqueous solution having a pH of between about 6 and about 8 (e.g., a pH of 6±1, 6.5±1, or 7±1), (ii) between about 5 mg/mL and 12 mg/mL (e.g., 6±1, 7±1, 8±1, 9±1, 10±1, or 11±1 mg/mL) arginine bumetanide salt, and (iii) and pharmaceutical excipients including a sugar or a sugar alcohol. In particular embodiments, the arginine bumetanide has a concentration of between about 8 mg/ml and about 10 mg/mL. In some embodiments, the aqueous solution has a pH of about 6.5 to about 7.5. In particular embodiments, the pharmaceutical composition includes benzyl alcohol (e.g., between about 0.2% (w/w) and about 1% (w/w) benzyl alcohol). In a particular embodiment, the pharmaceutical composition includes about 0.5% (w/w) benzyl alcohol.

In a related aspect, the invention features a pharmaceutical composition including (i) an aqueous solution having a pH of between about 6 and about 8 (e.g., a pH of 6±1, 6.5±1, or 7±1), (ii) between about 5 mg/ml and 12 mg/ml (e.g., 6±1, 7±1, 8±1, 9±1, 10±1, or 11±1 mg/mL) potassium bumetanide salt, and (iii) and pharmaceutical excipients including a sugar or a sugar alcohol. In particular embodiments, the potassium bumetanide has a concentration of between about 8 mg/ml and about 10 mg/mL. In some embodiments, the aqueous solution has a pH of about 6.5 to about 7.5. In particular embodiments the pharmaceutical composition includes benzyl alcohol (e.g., between about 0.2% (w/w) and about 1% (w/w) benzyl alcohol). In a particular embodiment, the pharmaceutical composition includes about 0.5% (w/w) benzyl alcohol.

In an embodiment of any of the above methods, the pharmaceutical composition includes mannitol (e.g., between about 2.5% (w/w) and about 5% (w/w) mannitol, or about 3±0.5%, 3.5±0.5%, 4±0.5%, or 4.5±0.5%, (w/w) mannitol).

In an embodiment of any of the above methods, the pharmaceutical composition includes sucrose (e.g., between about 1% (w/w) and about 3% (w/w) sucrose, or about 1.5±0.5%, 2.0±0.5%, or 2.5±0.5%, (w/w) sucrose).

### BRIEF DESCRIPTION OF THE DRAWINGS

FIG. 1 is a graph showing the solubility (mg/ml) of various bumetanide salt forms compared to the weight (mg) of the excipient that was added per 20 mg of bumetanide.

FIG. 2 is a graph showing the concentration (ng/ml) of bumetanide in the serum of dogs over a period of 0 to 240 minutes after IV or SC administration to compare the concentration of bumetanide achieved in serum using IV administration of 1.0 mg of commercially available bumetanide versus the concentration of bumetanide achieved with SC administration of 1.01 mg of the FS-3 and FS-4, 0.97 mg of the FS-5, or 1.0 mg of the FS-6 bumetanide formulations.

FIG. 3 is a graph showing the concentration (ng/ml) of bumetanide in the serum of dogs over a period of 0 to 60 minutes after IV or SC administration to compare the concentration of bumetanide achieved in serum using IV administration of 1.0 mg of commercially available bumetanide versus the concentration of bumetanide achieved with SC administration of 1.01 mg of the FS-3 and FS-4, 0.97 mg of the FS-5, or 1.0 mg of the FS-6 bumetanide formulations.

FIG. 4 is a graph showing the area under the curve (AUC) of the concentration of bumetanide in the plasma of dogs over a time period of 2 to 240 minutes (ng*h/mL) after IV administration of about 1.0 of commercially available bumetanide, subcutaneous administration of approximately 1.01 mg of the FS-4, 1.01 mg of the FS-4 formulation, 0.97 mg of the FS-5 formulation, 1.0 mg of the FS-6 formulation, and intranasal administration of approximately 1.0 mg of the F82 formulation.

### DEFINITIONS

As used herein, the term “about” refers to a value that is within 10% above or below the value being described. For instance, the phrase “about 6 mg/mL” refers to a value between 5.4 and 6.6 mg/mL.

As used herein, the term “effective amount,” refers to a quantity of a pharmaceutical composition sufficient to, when administered to the subject, for example a human subject, effect beneficial or desired results, such as clinical results. For example, in the context of edema, described herein, this term refers to an amount of the composition sufficient to achieve a reduction in the symptoms of edema as compared to the response obtained without administration of the composition. The quantity of a given composition described herein that will correspond to such an amount depending upon various factors, such as the given agent, the pharmaceutical formulation, the route of administration, the identity of the subject (e.g., age, sex, weight) being treated, and the like.

As used herein, “emulsion” is meant a two-phase colloidal system, such as a mixture of two or more immiscible liquids, which can be added to a pharmaceutical composition as an excipient. Liquid emulsions are those in which both the dispersed and the continuous phases are liquid. Energy input through shaking, stirring, homogenizing, or spray processes are typically needed to form an emulsion. For example, the emulsion can include an aqueous phase and a nonaqueous phase, and can include a self emulsifying system, or the emulsion can be nano-particulate containing an aqueous phase and a nonaqueous phase (e.g., a nanoemulsion or microemulsion). By “nanoemulsion or microemulsion” is meant a clear, stable, isotropic liquid mixture of oil, water, and surfactant, optionally in combination with a cosurfactant. The aqueous phase may contain salt(s) and/or other ingredients in addition to a biologically active agent. In contrast to ordinary emulsions, microemulsions form upon simple mixing of the components and do not require the high shear conditions generally used in the formation of ordinary emulsions. The two basic types of microemulsions are direct (oil dispersed in water, o/w) and reversed (water dispersed in oil, w/o).

As used herein, the terms “edema in the lung” or “pulmonary edema” refer to a condition where the patient has excess fluid in the lungs, which results in difficulty breathing. Edema in lungs may be caused by conditions related to heart failure, pneumonia, trauma, an allergic reaction, or another cause.

As used herein, the term “edema refractory to oral diuretics” refers to edema that is not responsive to oral diuretic treatment, such that diuresis is not achieved and excessive bodily fluid persists despite treatment with oral diuretics, thus resulting in persistence of the edematous state.

As used herein, the term “failed to achieve diuresis” refers to a patient's lack of increased voiding of bodily fluid and consequent persistence of an edematous state despite administration of oral diuretics.

As used herein, the term “loop diuretic” means a drug used in patients with congestive heart failure or renal insufficiency to reduce symptoms of hypertension and edema. A loop diuretic belongs to a class of diuretic agents that reduce reabsorption of sodium and chloride by the kidney leading in an increased secretion of urine.

As used herein, the term “low viscosity sodium carboxymethyl cellulose” or “low viscosity sodium CMC” refers to sodium carboxymethyl cellulose which has a viscosity of between 30 cP and 45 cP (e.g., 30±1 cP, 31±1 cP, 32±1 cP, 33±1 cP, 34±1 cP, 35±1 cP, 36±1 cP, 37±1 cP, 38±1 cP, 39±1 cP, 40±1 cP, 41±1 cP, 42±1 cP, 43±1 cP, 44±1 cP, and 45±1 cP) in 2% aqueous solution at 25° C. or between 50-200 cP in 4% aqueous solution at 25° C. Low viscosity sodium carboxymethyl cellulose can have a molecular weight of approximately 90 kDa.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response, and other problem complications commensurate with a reasonable benefit/risk ratio.

As used herein, the term “potassium-sparing diuretics” refers to a kind of diuretic drug that does not promote the secretion of potassium into urine, thus increasing fluid voiding. These diuretics can be used alone or in conjunction with loop or thiazide diuretics.

As used herein, the term “reduced intestinal motility” refers to a slowing of the activity of the gastrointestinal tract in a subject. One effect of the slowed activity can be reduced absorption, thus preventing effective absorption of pharmaceutical compositions. For example, this reduced intestinal motility can be caused by edema (e.g., such as fluid overload resulting from congestive heart failure) and prevents adequate absorption of the oral diuretics necessary for the treatment of the edema.

As used herein, the term “risk of hospitalization” refers to the potential likelihood that a patient is hospitalized for the treatment of edema, instead of effectively treating the edema without hospitalization by self-administering bumetanide using a method of the invention. A reduced risk of hospitalization is assessed for a given diseased population (e.g., patients suffering from congestive heart failure) of a particular severity comparing hospitalization rates for the treatment of edema in patients self-administering bumetanide to patients relying solely upon oral diuretics to treat the edema. Using the bumetanide methods of the invention, the hospitalization rates for the treatment of edema in a population of patients can be reduced by at least 10%, 20%, 30%, or 50%, and so the risk of hospitalization in individual patients using bumetanide can be reduced.

As used herein, “treatment” and “treating” refer to therapy for a subject in need of diuresis, such as therapy to ameliorate one or more symptoms of edema in a subject suffering from edema, or prophylactically reducing the risk of one or more symptoms of edema in a subject at risk of from edema.

As used herein, the term “unit dose” or “dosage” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle.

### DETAILED DESCRIPTION

The invention features pharmaceutical compositions and methods for the treatment of edema refractory to oral diuretics. Bumetanide can be formulated for subcutaneous delivery in salt forms that permit therapeutically effective amounts of bumetanide to be delivered in small volumes (about 100-150 μL) particularly suitable for subcutaneous delivery. It is optimal for the concentration of the bumetanide to by 5 mg/mL or 10 mg/mL such that an ideal dosage can be achieved in this small volume. Concentrations of the bumetanide salt in solution approach their limit of saturation as changes in solubility occurring as a result of change in temperature and storage conditions need to be considered to ensure suitable shelf-life stability of the pharmaceutical. This further emphasizes the need addressed by this invention for salt forms capable of stability at least 5 mg/mL.

With these concentrations of bumetanide salts, subcutaneous administration of bumetanide to patients who are experiencing edema refractory to oral diuretics due to their edematous state (e.g., an occurrence found in patients suffering from edema related reduced intestinal motility) can relieve the edema and restore the effectiveness of orally administered diuretics without the need for hospitalization. Subjects may be able to self-administer the pharmaceutical composition based on their symptoms, therefore, preventing the need for hospitalization, or the pharmaceutical composition may be administered by a medical professional (e.g., medical doctor, emergency responder or nurse) when a subject is experiencing acute distress.

Furthermore, the methods of the invention can reduce the risk of kidney failure in certain patients, such as those suffering from congestive heart failure. When such patients are in an edematous state and experiencing fluid overload, gastrointestinal absorption can be compromised, thus limiting the effectiveness of oral diuretics. As a result, the patient is often instructed to increase the dosage of oral diuretics putting further strain on the patient's kidneys. Eventually, oral diuretics become insufficient to remove excess fluid causing the patients to be hospitalized to receive intravenous diuretics to bypass the gastrointestinal system. Consequently, the patients are at risk of over-diuresis once gastrointestinal absorption is restored. This over-diuresis can result in kidney failure.

### Pharmaceutical Compositions

The bumetanide formulations of the invention can be solutions or suspensions. The formulations can include antioxidants, pH adjusting agents (e.g., an acid or a base), buffering agents, preservatives, tonicity agents, and/or viscosity enhancers (e.g., carboxymethylcellulose. The formulations may be administered as an aqueous solution on in the form of an emulsion, including nanoemulsions and microemulsions. The formulations may be provided in a single or multidose form. The formulation may be administered subcutaneously via, e.g., a pre-loaded syringe.

### Viscosity Enhancers

Viscosity enhancers can be used in the pharmaceutical compositions of the invention formulated for subcutaneous administration. Viscosity enhancers that can be used in accordance with the present invention include, without limitation, cellulose derivatives, carbomers (Carbopol), gums, and hyaluronic acids (hyaluronates), dextrans, polyvinyl alcohol, polyacrylic acids, povidone, polyethylene glycol, polyols (e.g., glycerol), propylene glycol and chitosans; where for cellulose derivatives particularly preferred are one or more of carboxymethyl cellulose (“CMC”) high molecular weight blend, CMC low molecular weight blend, CMC moderate molecular weight blend, Sodium CMC (low viscosity), methylcellulose, methyl cellulose 4000, hydroxymethyl cellulose, hydroxypropyl cellulose (“HPC”), hydroxypropylmethyl cellulose high molecular weight blend (“HPMC”), hydroxyl propyl methyl cellulose 2906, carboxypropylmethyl cellulose high molecular weight blend (“CPMC”), hydroxyethyl cellulose, or hydroxyethyl cellulose and hyaluronic acid. In particular embodiments, the viscosity enhancer is sodium CMC, preferably in combination with a polyol selected from the group consisting of mannitol, xylitol, sorbitol, isosorbide, erythritol, glycerol, maltitol and combinations thereof.

### Tonicity Agents

Tonicity agents can be utilized in the pharmaceutical compositions of the invention to modulate the tonicity of the liquid pharmaceutical composition. Tonicity in general relates to the osmotic pressure of a solution and is typically assessed relative to that of human blood serum. Tonicity agents may be included in pharmaceutical compositions (e.g., pharmaceutical dosage forms) to increase osmolality. Non-limiting examples of tonicity agents include substantially neutral buffering agents (e.g., phosphate buffered saline, tris buffer, or artificial perilymph), dextrose, mannitol, trehalose, sucrose, sorbitol, glycerin (aka glycerol), potassium chloride, and sodium chloride (e.g., as a hypertonic, isotonic, or hypotonic saline) as well amino acids (e.g., arginine, glycine, ornithine, lysine, histidine, glutamic acid, aspartic acid, isoleucine, leucine, alanine, phenylalanine, tyrosine, tryptophan, methionine, serine, and proline). Pharmaceutical compositions (e.g., pharmaceutical dosage forms) include sufficient amount of tonicity agents to provide for administration to a subject a hypertonic pharmaceutical dosage form.

### Buffering Agents

In some embodiments, the pharmaceutical composition includes a buffering agent. A buffering agent may be included in the pharmaceutical composition to increase stability of the composition by maintaining a consistent pH range. A buffering agent may improve the ability of the composition to be absorbed by the subject. Additionally, the buffering agent may act to increase the solubility of the composition. A buffering agent may increase the solubility of the drug by maintaining a high solubility of the drug at a constant pH range, preferably pH 6-8. In certain embodiments, the buffering agent maintains a pH range of between 6.5 and 6.7. The buffering agents that can be used in accordance with the present invention include but are not limited to, potassium hydroxide, arginine, and Lysine. One or more components of the pharmaceutical composition (e.g., potassium hydroxide, arginine, and Lysine) may act as the counter ion species of the buffer in which the bumetanide also functions as a buffering agent. The buffering agent may be citric acid, malic acid, succinic acid, potassium chloride, or ethylenediaminetetraacetic acid. For example, one or more buffering agents may be included in an amount of between about 5 mM and about 10 mM (e.g., 5±0.5 mM, 5.5±0.5 mM, 6±0.5 mM, 6.5±0.5 mM, 7±0.5 mM, 7.5±0.5 mM, 8±0.5 mM, 8.5±0.5 mM, 9±0.5 mM, 9.5±0.5 mM, or 10±0.5 mM) may be included in the pharmaceutical composition. A buffering agent may be added to the pharmaceutical composition depending on if the method of administration requires a specific pH range be maintained.

### Preservatives

In some embodiments of the pharmaceutical composition, a preservative is included as part of the pharmaceutical composition in order to increase the stability and/or shelf-life of the pharmaceutical composition. A preservative may be in the form of an antioxidant, an antimicrobial agent, or a chelating agent for example. An antioxidant may be added to the pharmaceutical composition in order to prevent the oxidation of other components in the composition that may be sensitive to oxidation in the presence of oxygen or sunlight. An antimicrobial agent may be included in the pharmaceutical composition in order to inhibit contamination of the pharmaceutical composition by microbes. A chelating agent may be added to the pharmaceutical composition to bind the pharmaceutically active ingredient to protect it from deterioration and increase stability. The preservatives that can be used in accordance with the present invention include, without limitation, benzyl alcohol, benzoic acid, and ethylenediaminetetraacetic acid (EDTA).

### Dosing Regimens

The dosing regimen used for the treatment methods described herein can vary depending on many factors, e.g., the age, health, and weight of the recipient; the nature and extent of the symptoms of edema; and the frequency and type of concurrent treatment, if any. One of skill in the art can determine the appropriate dosage based on the above factors. The compounds used in the methods described herein may be administered initially in a suitable dosage that may be adjusted as required, depending on the individual response. In general, a suitable dose of bumetanide according to the invention will be in the range of between 0.5 and 10 mg (e.g., 0.5, 1.0, 2.0, and 5 mg) of bumetanide in from 1 to 4 doses over a 1 to 4 hour period until the patient is no longer in an edematous state. Four doses of 100 μL per dose of the pharmaceutical composition may be delivered to the subject over a period of less than 1 hour. Alternatively, two doses of about 100 μL per dose may delivered to a subject followed by two more does of about 100 μL each after a period of about 30 to 60 minutes. The bumetanide can be administered to a patient at the onset of symptoms of edema. alternatively, the bumetanide is administered to a patient after symptoms persist following the failure of previously administered oral diuretics to treat the edema. If oral diuretics have been administered without effect, the patient may be restricted to an initial dose of less than 5 mg of bumetanide for a period of least 1 or 2 hours to further reduce the risk of over-diuresis, which may present as symptoms of dehydration and low blood pressure in the subject suffering from over-diuresis.

For the extended release formulation administered as a subcutaneous depot, the suitable dose of bumetanide according to the invention will be in the range of between 0.75 and 10 mg (e.g., 1.5±0.5, 2.5±0.5, 3.5±0.5, 4.5±0.5, 5.5±0.5, 6.5±0.5, 7.5±0.5, 8.5±0.5, and 9.5±0.5 mg) of bumetanide in from 1 to 2 doses over a 1 to 5 day period. The subcutaneous depot of bumetanide can be administered to a patient to prophylactically treat symptoms of edema and reduce the risk of rehospitalization.

### Oral Diuretics

The disclosure features methods and compositions for the treatment of edema refractory to oral diuretics. Oral diuretics have long been used to relieve fluid retention, a hallmark of congestive heart failure. Aggressive use of diuretics, even in people taking ACE inhibitors, can reduce hospitalizations and improve exercise capacity. Diuretics act on the kidneys to rid the body of excess salt and water. They reduce the accumulation of fluid in the legs, abdomen and lungs, lower blood pressure and improve the efficiency of the circulation. Side effects of diuretics include low blood pressure, dehydration, and kidney dysfunction; they also may trigger gout, increase blood sugar and triglyceride, LDL, and overall cholesterol levels and may deplete the B vitamin thiamin. Although many diuretics are available, they are generally categorized as thiazides and loop diuretics, used with or without potassium-sparing agents. It is important to note that a recent study found an increased incidence of hospitalization in patients who were taking nonsteroidal anti-inflammatory drugs (NTHEs) along with diuretics. Common NSAIDS include aspirin, ibuprofen, and naproxen. Thiazides, including hydrochlorothiazide (HydroDIURIL, Esidrix), chlorothiazide (Diuril), metolazone (Zaroxolyn), and chlorthalidone (Hygroton), are usually prescribed for patients with mild heart failure and good kidney function.

Loop diuretics, such as furosemide (Lasix), bumetanide (BumeX), and ethacrynic acid (Edecrine), are generally used for more severe heart failure, especially when kidney function is impaired. Loop diuretics are used intravenously to treat pulmonary edema and acute congestive heart failure, a thiazide and a loop diuretic may be administered simultaneously. Fluid may persist in the lungs even after standard treatment for congestive failure, limiting the patient's ability to function normally. One study treated patients with this condition very aggressively with furosemide to further reduce fluids, but no improvement was observed. Another method using a filtration technique was more successful.

Potassium loss is a major problem with diuretic use. Unless patients are also taking ACE inhibitors, which raise potassium levels, the physician may recommend a potassium supplement or the use of a potassium-sparing diuretic, such as spironolactone (Aldactone), amiloride (Midamor), and triamterene (Dyrenium), along with a thiazide or loop diuretic. All patients receiving diuretics with or without potassium-sparing drugs should have their blood potassium levels checked at regular intervals.

The methods of the invention can include the bumetanide therapy described herein used in combination with oral diuretics, such as loop diuretics, potassium-sparing diuretics, thiazides, or other oral diuretics, to manage edema in patients. Typically, the subcutaneously administered bumetanide is administered to a patient having taken oral diuretics previously, for example, within the past 1 hour, 2 hours, or 4 hours, for edema that is discovered to be refractory to oral diuretics. For example, the patient may be taking oral diuretics, such as high ceiling loop diuretics (e.g., furosemide, ethacrynic acid, torsemide and bumetanide), thiazides (e.g., hydrochlorothiazide acid), carbonic anhydrase inhibitors (e.g., acetazolamide and methazolamide), potassium-sparing diuretics (e.g., aldosterone antagonists: spironolactone, and epithelial sodium channel blockers: amiloride and triamterene), and/or calcium-sparing diuretics. The patient can continue with their regular regimen of oral diuretics following successful treatment of the acute edematous state using the methods of the invention.

### Selection of Subject

The methods and compositions of the invention can be used in patients generally at risk of edema. The edema may be, for example, pedal edema, peripheral edema, lung edema (e.g., pulmonary edema). The methods and compositions of the invention may be used where the subject is a mammal. Specifically, the subject may be a dog, where the dog is need of veterinary care. Additionally, the subject may be a human. Subjects that may be treated using the methods described herein are subjects having a diagnosis of congestive heart failure. Congestive heart failure (CHF) is characterized by an inability of the heart to generate sufficient cardiac output to meet the body's demands. Patients having CHF experience signs and symptoms of intravascular and interstitial volume overload, including shortness of breath, rapid heart rate, fluid in the lungs, and edema, along with indicators of inadequate tissue perfusion, including fatigue and/or poor exercise tolerance. Subjects that may be treated include those that have heart failure diagnosed by the standard and routine diagnostic procedures known in the art (e.g., electrocardiogramaction fraction), radionuclide imaging, magnetic resonance imaging, computed tomography imaging, cardiac catheterization with angiography, heart muscle biopsy, and/or assessment of atrial natriuretic peptide (ANP) and/or B-type natriuretic peptide levels in the blood (BNP)).

Subjects that may be treated are currently experiencing symptoms known to be associated with congestive heart failure, such as shortness of breath (e.g. dyspnea), fatigue, weakness, peripheral edema, or fluid overload. Subjects may be taking a daily dosage of oral diuretics, such as loop diuretics, potassium-sparing diuretics, or thiazides, to reduce the fluid overload and edema along with any one of the symptoms associated with congestive heart failure.

Subjects that may be treated using the methods described herein are experiencing symptoms of renal insufficiency caused by a reduction in blood flow, such as decreased urine output, swelling of the legs, ankles, feet, or abdomen, shortness of breath or fatigue caused by fluid overload as a result of the renal insufficiency. Subjects may experience fluid overload to the level wherein intestinal mobility becomes compromised preventing normal absorption of ingested nutrients, leading to inadequate gastrointestinal absorption, making for reduced bioavailability of pharmaceutical compositions administered orally.

Subjects that may be treated using the methods described herein can be experiencing symptoms of refractory edema as a result of congestive heart failure along with decreased intestinal mobility, thus preventing the subject's oral diuretics from effectively causing diuresis to remove excess bodily fluid. As a result of the persistence of symptoms of edema such as fatigue, shortness of breath and swollen limbs and abdomen, the subject seeks hospitalization to be administered intravenous diuretics, which then have the potential to put the subject at risk for kidney failure.

The subcutaneous administration of an extended release depot formulation can be used to prophylactically treat a patient and allow the patient to benefit from a reliable diuretic effect over a period of days without complications. The extended release therapy can be used to treat a patient at risk of rehospitalization. For example, following hospitalization for the treatment of congestive heart failure, the patient can receive treatment after discharge from the hospital, such that diuresis is achieved and symptoms such as shortness of breath, fatigue, and edema are relieved for an extended period of time (e.g., days).

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Solubility of Bumetanide in Water in the Presence and Absence of Surfactant

Bumetanide, 10 mg, in its free acid form was added to 1 mL of deionized water and mixed using a vortex and sonicator. The solution was filtered using a 0.22 μm nylon syringe filter. To 900 μL deionized water, 100 μL of the filtered bumetanide solution was added before being characterized for the concentration of the bumetanide in solution using HPLC. The concentration of bumetanide was determined using a standard curve generated by bumetanide solutions of known concentrations. Nearly no bumetanide in the free acid form was soluble water. Therefore, to the 10 mg of bumetanide, 1 mL of deionized water containing 1.0%, 0.5% or 0.1% of the surfactant tetradecyl-β-D-maltoside. The bumetanide was then solubilized using a vortex and sonicator, after which it was filtered using a 0.22 μm nylon syringe filter. To 900 μL deionized water, 100 μL of the filtered bumetanide solution was added before being characterized for the concentration of the bumetanide in solution using HPLC. The resulting bumetanide concentrations are recorded in Table 1.

TABLE 1
Measured solubility of
bumetanide with a non-ionic surfactant

| | Calculated | | | Amount |
|---|---|---|---|---|
| | conc. | | | added |
| Sample | (mg/mL) | pH | Surfactant added | (% wt) |
| F39 | 0.01 | 4 | Tetradecyl-β-D-Maltoside | 0.1% |
| F40 | 0.08 | 4 | (non-ionic solubilizer) | 0.5% |
| F41 | 0.21 | 4 | | 1.0% |

While addition of the surfactant, did improve the amount of bumetanide able to be solubilized in water, these results clearly show that when bumetanide is not in a salt form, it has incredibly low solubility in water despite the presence of a surfactant.

### Example 2. Solubility of Bumetanide Salts

The acid form of bumetanide was weighed out to 20 mg and dissolved in 1 mL of deionized water by mixing with a vortex to form a low-pH solution of bumetanide. To this low pH bumetanide solution, a determined amount of base was added and mixed using a vortex for 2 minutes, to form a solution having a pH suitable for administration. With the increasing pH the bumetanide salt formed in situ precipitated, in part, to form a saturated solution of the resulting salt form. The saturated solution was then filtered through a 0.22 μm filter to remove all precipitated bumetanide salt. The filtrate was collected, and its pH was measured. An aliquot of the saturated solution was assayed for bumetanide by HPLC to determine the maximum concentration achievable for the salt form in water at about neutral pH.

This procedure was repeated for various bases, including arginine, lysine, potassium, sodium, glycine, histidine, and the cationic lipophilic surfactants soy lecithin, distearoyl glycerol-3-phosphatidylamine, L-α-phosphatidylethanolamine and bis(2-ethylhexyl)amine, in order to determine the maximum solubility of each bumetanide salt solution. The concentration of bumetanide in solution for each bumetanide salt form is recorded in Table 2 in order to determine the maximum solubility of each bumetanide salt.

TABLE 2
Measured solubility of bumetanide salt with various bases

| | | | | | Bu- |
|---|---|---|---|---|---|
| | | | | | meta- |
| | Calcu- | | | | nide: |
| Sample | lated | pH | Base added | Base | Base |
| F22 | 8.94 | 6.98 | Arginine | 4.94 | |
| F23 | 5.65 | 6.94 | MW 174.2 | 5.14 | |
| F24 | 11.65 | 7.04 | | 5.84 | |
| F25 | 12.60 | 7.10 | | 7.94 | (1.1:1) |
| F26 | 5.89 | 6.87 | Lysine | 3.94 | |
| F27 | 4.56 | 6.85 | MW 146.19 | 5.12 | |
| F28 | 6.07 | 6.83 | | 6.00 | |
| F29 | 7.08 | 6.93 | | 6.93 | (1.1:1) |
| F30 | 7.75 | 7.14 | KOH | 1.12 | |
| F31 | 14.04 | 7.13 | MW 56.11 | 2.24 | |
| F32 | 16.97 | 7.23 | | 2.81 | (1:1) |
| F33 | 20.19 | 7.27 | | 3.37 | (1:1.1) |
| F34 | 5.67 | 7.03 | NaOH | 0.80 | |
| F35 | 5.58 | 6.99 | MW 39.998 | 1.60 | |
| F36 | 5.62 | 9.39 | | 2.00 | |
| F37 | 6.59 | 10.19 | | 2.40 | (1:1) |
| F45 | 0.03 | 5.11 | Glycine | 4.14 | (1:1) |
| F46 | 0.04 | 5.16 | MW 75.1 | 6.28 | |
| F47 | 0.05 | 5.15 | | 8.34 | |
| F48 | 0.06 | 5.18 | | 10.06 | (1:2) |
| F49 | 2.04 | 6.47 | Histidine | 4.04 | |
| F50 | 3.42 | 6.50 | MW 155.2 | 5.02 | |
| F51 | 3.23 | 6.62 | | 6.09 | |
| F52 | 3.52 | 6.68 | | 8.03 | (1:1) |
| F53 | 0.25 | 4.56 | Soy Lecithin | 10.14 | |
| F57 | 0.00 | 6.05 | Distearoyl glycerol-3- | 20.87 | |
| F58 | 0.00 | 6.03 | phosphatidylamine | 25.78 | |
| F59 | 0.00 | 5.05 | MW 748.1 | 32.6 | |
| F60 | 0.00 | 4.54 | | 41.78 | (1:1) |
| F71 | 0.01 | 4.07 | L-α- | 4.98 | |
| F72 | 0.01 | 3.97 | Phosphatidylethanolamine | 6.02 | |
| F73 | 0.01 | 3.68 | (di-ionic surfactant) | 7.97 | |
| F74 | 0.00 | 3.72 | | 9.99 | |
| F75 | 0.19 | 5.45 | Bis(2-ethylhexyl)amine | 6.47 | |
| F76 | 0.19 | 5.51 | MW 241.61 | 8.31 | |
| F77 | 0.18 | 5.79 | | 9.83 | |
| F78 | 0.18 | 5.8 | | 12.98 | (1:1) |

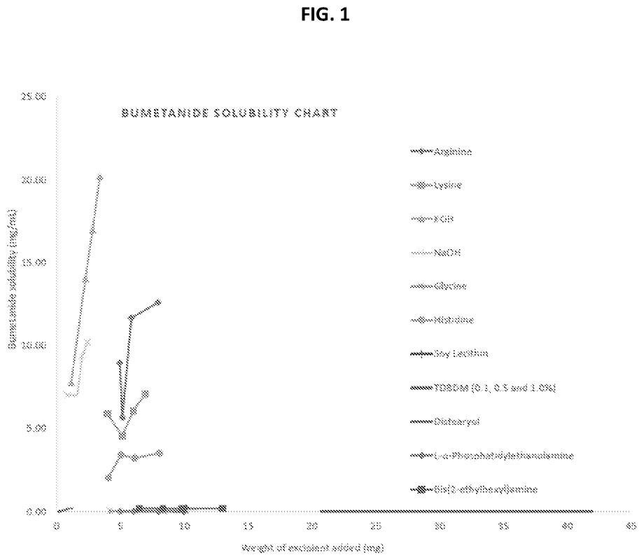

This solubility data clearly shows the highest concentration of bumetanide salt in solution is achieved when either arginine or potassium are acting as the base with their greatest concentrations measured to be 12.6 mg/ml and 20.19 mg/mL respectively. The solubility of the bumetanide salt is significantly increased when potassium hydroxide acting as the base as opposed to another base such as sodium hydroxide which has a measured solubility of 6.59 mg/mL, as is shown in FIG. 1. It is surprising that the solubility of the bumetanide salt is significantly increased when arginine is acting as the base as opposed to another amino acid base such as lysine, which has a measured solubility of 7.08 mg/ml. As shown in FIG. 1, the potassium salt of bumetanide achieves the highest solubilities at pharmaceutically desirable pHs (e.g., pH 6-8) with the lowest mass burden to the transmucosal formulation, resulting in concentrations that (as shown in the animal studies) can produce a PK performance similar to that produced with IV injection without the complications and risks associated with IV administration.

### Example 3. Solubility of Bumetanide in Different pH Buffers

The solubility of bumetanide was assessed in various pH buffers. 10 mg of bumetanide was weighed into a 3 mL glass vial. To the vial, 2 mL of buffer with a desired pH was added. The solution was then sonicated for 2 hours at room temperature (25° C.) and then rotated overnight. The solution was then filtered through a 0.2 μm nylon syringe filter, and the first 0.5 mL was discarded. The filtrate was then diluted 10× and at least 0.5 mL was aliquoted into an HPLC vial. 1 M sodium citrate was used for F1 to F4, and 1 M sodium phosphate buffer was used for F5 to F9. The resulting concentration of bumetanide in solution was measured for each pH buffer and the results are summarized in Table 3.

TABLE 3
Solubility of Bumetanide in solution in various pH buffers

| | | | | | | Conc.(μg/mL) |
|---|---|---|---|---|---|---|
| | | | | Avg | | (API solubility in |
| Sample | pH | Inj#1 | Inj#2 | PA | R.T. (min) | buffer solutions) |
| F1 | 4.0 | N/D | N/D | — | — | — |
| F2 | 4.5 | 18.2 | 19.5 | 18.9 | 11.79 | 7.2 |
| F3 | 5.0 | 87.0 | 89.4 | 88.2 | 11.80 | 33.8 |
| F4 | 5.5 | 180.8 | 184.3 | 182.6 | 11.80 | 69.9 |
| F5 | 6.0 | 83.4 | 84.9 | 84.2 | 11.81 | 32.2 |
| F6 | 6.5 | 157.2 | 159.6 | 158.4 | 11.81 | 60.6 |
| F7 | 7.0 | 356.3 | 360.8 | 358.6 | 11.81 | 137.3 |
| F8 | 7.5 | 564.0 | 565.9 | 565.0 | 11.82 | 216.3 |
| F9 | 8.0 | 760.1 | 752.6 | 756.4 | 11.81 | 289.6 |

1 g of Kolliphor RH40 and 9 g of ethanol was added to a 15 mL Falcon tube. The solution was mixed and vortexed as needed. 50 mg of bumetanide salt was weighed out and transferred into the solution and mixed, vortexed, or sonicated as needed to dissolve solids to form a 5 mg/g stock solution. If all solids dissolved, 10 more mg of bumetanide salt was added. 1.0 g of this stock solution was added to a 10 mL glass vial using a 0.22 μm nylon syringe filter. The pH of the solution was measured and adjusted with 0.1 N HCl or NaOH such that was within the target pH by +/−0.1. If there was no precipitate in the vial, 0.5 mL of each formulation was aliquoted into a 1.5 mL HPLC vial for a total of 20 vials. If there was precipitate, the solution was transferred with the precipitate into a 10 ml syringe and filtered through a 0.45 μm filter. The filtrate was collected and aliquoted into 0.5-1.0 mL of each formulation in a 1.5 mL HPLC vial for total 15 vials. Each vial was crimp sealed. 4 vials were placed at 40° C., 4 vials were placed at 50° C., and 4 vials were placed at 60° C. 2 vials were placed at −80° C. for back up. The last vial was used for TO assay analysis. The pH and concentration of the various formulations was measured on the day of, 3 days, 7 days, and 10 days of being stored at the indicated temperatures. The results are summarized in Table 4. The percent bumetanide recovery was also measured on these days, and the results are summarized in Table 5.

TABLE 4
Solubility of Bumetanide in solution in various
pH buffers at variable temperature
T0 assay:

| Sample | Inj#1 | Inj#2 | Avg PA | Conc.(mg/g) |
|---|---|---|---|---|
| F5 pH 6.1 | 13643.6 | 13620.1 | 13631.9 | 5.23 |
| F6 pH 6.5 | 12914.5 | 12956.2 | 12935.4 | 5.24 |
| F7 pH 7.0 | 14852.8 | 14814.2 | 14833.5 | 5.74 |
| F8 pH 7.6 | 15101.9 | 15050.4 | 15076.2 | 5.79 |
| F9 pH 8.0 | 15108.2 | 15095.6 | 15101.9 | 5.90 |

| | 25° C. | | 40° C. | | 50° C. | | 60° C. | |
|---|---|---|---|---|---|---|---|---|
| | | Conc. | | Conc. | | Conc. | | Conc. |
| Sample | pH | (mg/g) | pH | (mg/g) | pH | (mg/g) | pH | (mg/g) |

T 3 DAY assay:

| | | | | | | | | |
|---|---|---|---|---|---|---|---|---|
| F5 | 7.0 | 5.78 | 7.5 | 5.84 | 7.5 | 5.83 | 7.5 | 5.78 |
| F6 | 6.8 | 5.92 | 6.9 | 5.99 | 6.9 | 5.96 | 6.9 | 5.92 |
| F7 | 7.5 | 5.92 | 7.7 | 5.91 | 7.8 | 5.85 | 7.8 | 5.87 |
| F8 | 7.9 | 5.88 | 8.2 | 5.85 | 8.3 | 5.81 | 8.2 | 5.81 |
| F9 | 8.5 | 5.98 | 8.5 | 5.95 | 8.4 | 5.92 | 8.3 | 5.92 |

T 7 DAY assay:

| | | | | | | | | |
|---|---|---|---|---|---|---|---|---|
| F5 | 7.2 | 5.79 | 7.2 | 5.82 | 7.3 | 5.82 | 7.3 | 5.79 |
| F6 | 6.8 | 5.99 | 6.8 | 5.94 | 6.9 | 5.95 | 6.9 | 5.96 |
| F7 | 7.6 | 5.90 | 7.8 | 5.88 | 7.7 | 5.89 | 7.8 | 5.87 |
| F8 | 8.1 | 5.79 | 8.1 | 5.80 | 8.1 | 5.79 | 8.0 | 5.85 |
| F9 | 8.2 | 5.94 | 8.3 | 5.90 | 8.3 | 5.87 | 8.2 | 5.90 |

T 10 DAY assay

| | | | | | | | | |
|---|---|---|---|---|---|---|---|---|
| F5 | 7.2 | 5.82 | 7.2 | 5.82 | 7.3 | 5.80 | 7.3 | 5.85 |
| F6 | 6.8 | 5.98 | 6.8 | 5.98 | 6.8 | 5.91 | 6.8 | 5.98 |
| F7 | 7.6 | 5.90 | 7.6 | 5.88 | 7.5 | 5.85 | 7.6 | 5.90 |
| F8 | 7.9 | 5.85 | 8.0 | 5.77 | 8.0 | 5.80 | 7.9 | 5.84 |
| F9 | 8.2 | 5.92 | 8.1 | 5.91 | 8.0 | 5.85 | 7.8 | 5.92 |

TABLE 5
Percent recovery of bumetanide at various temperatures over time

| | | 25° C. | 40° C. | 50° C. | 60° C. |
|---|---|---|---|---|---|
| | | Assay | Assay | Assay | Assay |
| | | Recovery | Recovery | Recovery | Recovery |
| | Time | % | % | % | % |
| Sample | (day) | over T0 | over T0 | over T0 | over T0 |
| F5* | 0 | — | — | — | — |
| | 3 | 110.6 | 111.6 | 111.4 | 110.5 |
| | 7 | 110.8 | 111.2 | 111.2 | 110.8 |
| | 10 | 111.3 | 111.2 | 110.8 | 111.9 |
| F6* | 0 | — | — | — | — |
| | 3 | 112.9 | 114.3 | 113.8 | 113.0 |
| | 7 | 114.2 | 113.3 | 113.4 | 113.8 |
| | 10 | 114.0 | 114.0 | 112.8 | 114.1 |
| F7 | 0 | — | — | — | — |
| | 3 | 103.2 | 102.9 | 102.0 | 102.3 |
| | 7 | 102.8 | 102.4 | 102.5 | 102.3 |
| | 10 | 102.7 | 102.4 | 102.0 | 102.8 |
| F8 | 0 | — | — | — | — |
| | 3 | 101.5 | 101.0 | 100.4 | 100.4 |
| | 7 | 100.1 | 100.2 | 100.0 | 101.1 |
| | 10 | 101.1 | 99.6 | 100.1 | 100.9 |
| F9 | 0 | — | — | — | — |
| | 3 | 101.4 | 101.0 | 100.4 | 100.5 |
| | 7 | 100.7 | 100.1 | 99.6 | 100.1 |
| | 10 | 100.4 | 100.3 | 99.2 | 100.4 |

### Example 4. Emulsion Formulations of Bumetanide Salts

Emulsion formulations of bumetanide salts were developed. Glycerol (2.25% weight), medium chain triglycerides (MCT) (10% weight) and lecithin (E-80) (1.2% weight) were weighed out and dissolved by way of sonication for 30 minutes, which was followed by addition of 86% weight deionized water and additional mixing until a clear and colorless solution was obtained to generate the FEV-1 product described in Table 6. FEV-2 was generated by weighing out MCT and lecithin into a tared 50 mL falcon tube and sonicated for about 30 min at 50° C. to ensure all solid dissolved. DI-water was mixed in with 10 the solution and a white emulsion was obtained, as described in Table 6.

TABLE 6
Composition and compounding of FEV-1 and FEV-2

| | FEV-1 | FEV-1 | FEV-2 | FEV-2 |
|---|---|---|---|---|
| Ingredient | (%, wt) | (mg/10 g) | (%, wt) | (mg/10 g) |
| MCT | 10 | 1000 | 10 | 1000 |
| Glycerol | 2.25 | 225 | — | — |
| Lecithin (E-80) | 1.2 | 120 | 1.2 | 120 |
| DI-water | 86.0 | 8600 | 88.8 | 8880 |

The FE-V1 product was used to prepare emulsions FE-1-FE-7 with various emulsifying agents. The FE-V2 product was used to prepare the FE-8 emulsion. These emulsions were prepared by weighing out bumetanide, an emulsifying agent (e.g. polysorbate 80, polysorbate 20 or PEG 400), and benzyl alcohol in the cases of FE-3-FE-6 as are described in Table 7. Each emulsion formulation was brought to 1000 mg using the FEV-1 solution and BB for 30 seconds for each formulation. The appearance is checked visually, and the pH is measured. For FE-6, the pH was adjusted to 5.7 using 1N NaOH, and for FE-7, the pH was adjusted to 6.5 using 1N NaOH. Each emulsion formulation was then filtered using a 0.22 μm filter syringe. The resulting filtrate was characterized using HPLC for their respective bumetanide concentrations as well as for any impurities.

TABLE 7
Compositions for formulations FE-1 to FE-7

| | | %, wt | | | | | | | |
|---|---|---|---|---|---|---|---|---|---|
| Ingredient | FE-1 | FE-2 | FE-3 | FE-4 | FE-5 | FE-6 | FE-7 | FE-8 | FE-9 |
| Bumetanide | 0.5 | 0.5 | 1.0 | 1.0 | 1.0 | 0.6 | 0.6 | 0.5 | 0.5 |
| Polysorbate | 5.0 | — | 5.0 | 8.0 | 8.0 | 8.0 | — | 8.0 | — |
| 80 | | | | | | | | | |
| Polysorbate | — | 5.0 | — | — | — | — | — | — | — |
| 20 | | | | | | | | | |
| PEG 400 | — | — | 10.0 | — | 10.0 | — | — | — | — |
| Benzyl | — | — | 0.5 | 0.5 | 0.5 | 0.5 | 0.5 | 0.5 | — |
| alcohol | | | | | | | | | |
| FEV-1 | 94.5 | 94.5 | 83.5 | 90.5 | 80.5 | 90.9 | 98.9 | — | 99.5 |
| FEV-2 | — | — | — | — | — | — | — | 100 | |
| pH | As is | As is | As is | As is | As is | 5.7 | 6.5 | | 6.1 |

The measured concentrations of bumetanide showed the highest concentrations were obtained with the emulsion formulation FE-6 with a bumetanide concentration of 6.55 mg/g as shown in Table 8.

TABLE 8
Measured bumetanide concentrations for emulsion compositions

| ID | FE-1 | FE-2 | FE-3 | FE-4 | FE-5 | FE-6 | FE-7 |
|---|---|---|---|---|---|---|---|
| Appearance | Translucent | Translucent | Milky | Milky | Milky | Milky | Clear |
| | sol. | sol. with | liquid | liquid | liquid | solution | solution |
| | | PPT | | | | | with |
| | | | | | | | PPT |
| Filterable | Yes | Yes | Yes | Yes | Yes | Yes | Yes |
| through | | | | | | | |
| 0.22 μm | | | | | | | |
| filter | | | | | | | |
| pH | 4.11 | 3.86 | 3.23 | 2.92 | 3.07 | 5.74 | 6.46 |
| Conc. | 3.79 | 4.50 | 2.07 | 3.02 | 2.86 | 6.55 | 3.60 |
| (mg/g) | | | | | | | |

To test stability of FE-9 at various temperatures, FE-9 was filled into spray vials and were placed in a stability chamber at 2-8° C., vials were placed in a stability chamber at 25° C., and vials were placed at −20° C. A remaining vial was used to measure initial concentration and impurities. After 1 week, 2 weeks, and 4 weeks, a sample was removed from the stability chambers and their appearance was checked visually. The samples were then prepared for HPLC for assay and impurity analysis. At this time, the pH and osmolality of the formulation was measured. The osmolality was at TO was 264 Osm and the viscosity was 1.3 cp, conducted at 10 rpm. The results of the stability study are summarized in Table 9.

TABLE 9
FE-9 Stability Study

| | | | | | | Sample | QS to | HPLC | Calculated | Recovery |
|---|---|---|---|---|---|---|---|---|---|---|
| Time | | | PA of | PA of | Average | Size | Volume | conc. | conc. | vs. T0 |
| (weeks) | Sample | pH | Inj. 1 | Inj. 2 | PA | (mL) | (mL) | (mg/mL) | (mg/g) | (%) |
| 0 | FE-9, T0 | 6.1 | 11571.3 | 11544 | 11557.7 | 0.518 | 5 | 0.48 | 4.59 | 100.0 |
| 1 | FE-9, | 6.1 | 11468.4 | 11462.9 | 11465.7 | 0.501 | 5 | 0.47 | 4.69 | 102.2 |
| | 2-8° C. | | | | | | | | | |
| | FE-9, 25° C. | 6.1 | 11383.5 | 11287.4 | 11335.5 | 0.504 | 5 | 0.46 | 4.60 | 100.3 |
| 2 | FE-9, | 6.1 | 11270.0 | 11256.1 | 11263.1 | 0.497 | 5 | 0.46 | 4.62 | 100.8 |
| | 2-8° C. | | | | | | | | | |
| | FE-9, 25° C. | 6.1 | 11742.7 | 11743.8 | 11743.3 | 0.518 | 5 | 0.48 | 4.63 | 100.9 |
| 4 | FE-9, | 6.1 | 11225.1 | 11155.4 | 11190.3 | 0.501 | 5 | 0.46 | 4.59 | 100.1 |
| | 2-8° C. | | | | | | | | | |
| | FE-9, 25° C. | 6.1 | 11354.8 | 11333.8 | 11344.3 | 0.494 | 5 | 0.47 | 4.72 | 102.9 |

Particle size distribution analysis was performed to determine the average particle size for the FE-9 bumetanide emulsion was less than 200 nm using dynamic light scattering on a Malvern Panalytical Zetasizer. The FE-9 sample was prepared for analysis by performing two passes of the emulsion through a microfluidizer prior to particle size distribution analysis. This process was repeated if measured average particle size was greater than 200 nm. The average particle sizes for FE-9 stored both at room temperature and 2-8° C. over a period of 4 weeks were 112-120 nm as described in Table 11.

TABLE 10
Particle size analysis for FE-9 emulsion

| | T | Z-Ave | | D(v, | D(v, | D(v, |
|---|---|---|---|---|---|---|
| Sample Name | (° C.) | (d · nm) | Pdl | 0.1) | 0.5) | 0.9) |
| FE-9_T0 | 25 | 112.0 | 0.173 | 41.4 | 74.8 | 227.0 |
| FE-9_2-8° C. 1 week | 25 | 112.5 | 0.140 | 53.9 | 87.9 | 216.0 |
| FE-9_25° C. 1 week | 25 | 115.0 | 0.117 | 59.2 | 94.0 | 212.0 |
| FE-9_2-8° C. 2 weeks | 25 | 118.4 | 0.111 | 62.6 | 99.4 | 217 |
| FE-9_25° C. 2 weeks | 25 | 119.5 | 0.127 | 58.3 | 94.5 | 237 |
| FE-9_2-8° C. 4 weeks | 25 | 118.3 | 0.126 | 62 | 99.5 | 220 |
| FE-9_25° C. 4 weeks | 25 | 119.8 | 0.122 | 60.6 | 97.1 | 234 |

FE-8 was prepared by weighing out each excipient into a tared 15 mL Falcon tube (see Table 7), and brought to QS with FEV-2. The pH was adjusted to 6.0 with 1N NaOH and vortexed to ensure no solid was in the mixture; sonication was used if necessary. The final pH was kept at 6.0 and the visual appearance was checked, pH was measured, and osmolality was measured. The solution was filtered through a 0.22 μm filter, and the filtrate was collected, and 1.0 mL was aliquoted into a 3 mL glass vial, which were crimped to seal the vial for total 8 vials.

To test stability at various temperatures, 3 vials were placed in a stability chamber at 2-8° C., 3 vials were placed in a stability chamber at 25° C., and 2 vials were placed at −20° C. The remaining vial was used to measure initial concentration and impurities. After 4 weeks, the samples were removed from the stability chambers and their appearance was checked visually. The samples were then prepared for HPLC for assay and impurity analysis. At this time, the pH and osmolality of the formulation was measured. Before the osmolality was measured, the 0.2 mL of the formulation was diluted with 1.8 mL of DI-water. The resulting concentration of bumetanide and the percent recovery for the samples stored at the various temperatures after 2 weeks is summarized in Table 9 and the osmolality was measured to be 5 mOsm with a dilution factor of 10 at TO.

TABLE 11
FE-8 Stability Test after 2 weeks

| | | | | Sample | QS to | HPLC | Calculated | Recovery |
|---|---|---|---|---|---|---|---|---|
| | PA of | PA of | Average | Size | Volume | conc. | conc. | vs. T0 |
| Sample | Inj. 1 | Inj. 2 | PA | (mL) | (mL) | (mg/mL) | (mg/g) | (%) |
| FE-8, −20° C. | 5763.8 | 5709.9 | 5736.9 | 0.25 | 5 | 0.24 | 4.76 | 102.0 |
| FE-8, 2-8° C. | 5831.4 | 5817.3 | 5824.4 | 0.25 | 5 | 0.24 | 4.78 | 102.4 |
| FE-8, 25° C. | 5711.1 | 5685.5 | 5698.3 | 0.25 | 5 | 0.23 | 4.67 | 100.1 |

### Example 5. Nano-Emulsion Formulations for Bumetanide Salts

Nano-emulsion formulations of bumetanide salts were developed to improve mucosal absorption. Nano-emulsions were prepared by first generating the FNEV-1 formulation by weighing out glycocholic acid (46.8 mg) and suspending in sterile water for injection (SWFI) (600 mg) using a vortex as described in Table 12. To this solution, 10 N NaOH was added (10 μL) and mixed until the solution became clear. Soy lecithin (PL90G) and benzyl alcohol were added as described in Table 12 and dissolved completely using a vortex and a sonicator if necessary. The solution was brought to a final weight with deionized water as described in Table 12 and mixed. The pH of this solution was measured and adjusted to a final pH of 7.0 after which the solution was allowed to shaker overnight to generate the FNEV-1 product.

TABLE 12
Composition and compounding of FNEV-1

| | FNEV-1 | |
|---|---|---|
| Excipient | Mg/g | 20 g Batch amount (mg) |
| Bumetanide | 0 | 0 |
| Soy lecithin (PL90G) | 100 | 2000 |
| Glycocholic acid | 46.8 | 936 |
| Benzyl alcohol | 9 | 180 |
| NaOH (10N) adjust pH to 7.0 | | |
| DI-H2O Q.S. to | 1 g | 20000 |

The FNEV-1 product was used to prepare the FNE-1 nano-emulsions with various pH values. The bumetanide nano-emulsions were prepared by weighing out bumetanide and dissolving in the FNEV-1 solution, as described in Table 13, using a vortex, and adjusting the pH to 5.0 or 6.0 using 1.0 N NaOH or 1.0 N HCl. The solutions were left standing overnight, after which the pH was measured. The solutions were filtered using a 0.22 μm centrifuge tube filter. The filtrates were then diluted by a factor of 20 and mixed using a vortex such that 0.25 mL of filtrate was diluted to a final volume of 5 mL before transferring the solution to the HPLC.

TABLE 13
Composition of nano-emulsion FNE-1

| Ingredient | mg/g | 100 g Batch amount (g) |
|---|---|---|
| Bumetanide | 5 | 0.509* |
| FNEV-1 | 995 | 99.5 |

| | |
|---|---|
| Dextrose | adjust osmolarity to about 290 |

*After taking Bumetanide purity (98.3%) into consideration.

The concentration of bumetanide in the FNE-1 solutions having final pH values of 5.3, 5.6, and 6.1 were measured using HPLC by comparison to solutions of known bumetanide concentration as shown in Table 14. The FNE-1 formulation with a pH of 6.1 showed the highest concentration of bumetanide in solution with a calculated concentration of 10.1 mg/mL. The results show the concentration of bumetanide in solution more than doubles when the pH of the nano-emulsion is changed from 5.3 to 6.1, thus, indicating that a pH of about 6 is optimal for concentrations of bumetanide in solution that are 5 mg/mL or greater.

TABLE 14
Measured bumetanide concentrations for FNE-1 with various pH values

| | | | | | Sample | QS to | HPLC | Calc. |
|---|---|---|---|---|---|---|---|---|
| | | PA of | PA of | Average | Size | Volume | conc. | conc. |
| Sample | pH | Inj. 1 | Inj. 2 | PA | (mL) | (mL) | (mg/mL) | (mg/g) |
| FNE-1, pH 5.3 | 5.3 | 3740.6 | 3733.8 | 3737.2 | 0.25 | 5 | 0.16 | 3.1 |
| FNE-1, pH 5.6 | 5.6 | 5579.3 | 5591.5 | 5585.4 | 0.25 | 5 | 0.23 | 4.7 |
| FNE-1, pH 6.1 | 6.1 | 12005 | 11957.4 | 11981.2 | 0.25 | 5 | 0.50 | 10.1 |

Particle size distribution analysis was performed to determine the average particle size for the FNE-1 bumetanide nano-emulsion using dynamic light scattering on a Malvern Panalytical Zetasizer. The average particle sizes for FNE-1 with pH 6.0 stored both at room temperature and 2-8° C. over a period of 4 weeks were 9-11 nm as described in Table 15.

TABLE 15
Particle size analysis of FNE-1 nano-emulsion

| Sample Name | T (° C.) | Z-Ave (d · nm) | Pdl | D(v, 0.1) | D(v, 0.5) | D(v, 0.9) |
|---|---|---|---|---|---|---|
| FNE-1_2-8 C. 1 week | 25 | 9.883 | 0.544 | 1.01 | 1.41 | 2.29 |
| FNE-1_25 C. 1 week | 25 | 11.27 | 0.609 | 0.98 | 1.35 | 2.23 |
| FNE-1_2-8 C. 2 weeks | 25 | 9.681 | 0.535 | 0.93 | 1.30 | 2.18 |
| FNE-1_25 C. 2 weeks | 25 | 8.924 | 0.496 | 1.07 | 1.43 | 2.21 |
| FNE-1_2-8 C. 4 weeks | 25 | 10.5 | 0.573 | 0.981 | 1.34 | 2.19 |
| FNE-1_25 C. 4 weeks | 25 | 8.661 | 0.483 | 1.03 | 1.41 | 2.24 |

To test stability at various temperatures, filled spray vials were placed in a stability chamber at 2-8° C., vials were placed in a stability chamber at 25° C., and vials were placed at −20° C. A remaining vial was used to measure initial concentration and impurities. After 1 week, 2 weeks, and 4 weeks, the samples were removed from the stability chambers and their appearance was checked visually. The samples were then prepared for HPLC for assay and impurity analysis; at this time, the pH was also measured. The osmolality of the formulation was measured and found to be 140 Osm after a two-fold dilution, and the viscosity was 6 cp conducted at 10 rpm. The resulting concentration of bumetanide and the percent recovery for the samples stored at the various temperatures over 4 weeks is summarized in Table 16.

TABLE 16
FNE-1 Stability Test Over 4 weeks

| | | | | | | Sample | QS to | HPLC | Calculated | Recovery |
|---|---|---|---|---|---|---|---|---|---|---|
| Time | | | PA of | PA of | Average | Size | Volume | conc. | conc. | vs. T0 |
| (weeks) | Sample | pH | Inj. 1 | Inj. 2 | PA | (mL) | (mL) | (mg/mL) | (mg/g) | (%) |
| 0 | FNE-1, | 6.0 | 12335.2 | 12286.4 | 12310.8 | 0.499 | 5 | 0.51 | 5.09 | 100 |
| | T0 | | | | | | | | | |
| 1 | FNE-1, | 6.0 | 12321.9 | 12415.2 | 12368.6 | 0.503 | 5 | 0.51 | 5.11 | 100.3 |
| | 2-8° C. | | | | | | | | | |
| | FNE-1, | 6.0 | 11999.7 | 11917.6 | 11958.7 | 0.488 | 5 | 0.50 | 5.09 | 99.9 |
| | 25° C. | | | | | | | | | |
| 2 | FNE-1, | 6.0 | 12723.7 | 12706.2 | 12715.0 | 0.508 | 5 | 0.52 | 5.08 | 99.8 |
| | 2-8° C. | | | | | | | | | |
| | FNE-1, | 6.0 | 12170.4 | 12107.1 | 12138.8 | 0.495 | 5 | 0.49 | 4.99 | 98.0 |
| | 25° C. | | | | | | | | | |
| 4 | FNE-1, | 6.0 | 12404.3 | 12466.2 | 12435.3 | 0.504 | 5 | 0.51 | 5.06 | 99.4 |
| | 2-8° C. | | | | | | | | | |
| | FNE-1, | 6.0 | 12091.9 | 12016.5 | 12054.2 | 0.497 | 5 | 0.49 | 4.98 | 97.7 |
| | 25° C. | | | | | | | | | |

### Example 6. Stability of Bumetanide Salts

The stability of the arginine and potassium bumetanide salts was monitored over a four week period with storage at either 2-8° C. or 25° C. for that time period. The bumetanide salt solutions were evaluated on the basis of pH, concentration of bumetanide in solution, the percent assay recovery and the presence of an impurity (see Tables 17 and 18).

TABLE 17
Stability measurements of arginine bumetanide salt

| Measuring | T0 | 1 week | | 2 weeks | | 4 weeks | |
|---|---|---|---|---|---|---|---|
| pH | 7.0 | 5° C. | 25° C. | 5° C. | 25° C. | 5° C. | 25° C. |
| | | 7.0 | 7.1 | 7.0 | 7.0 | 7.0 | 7.1 |
| Assay | 5.07 | 5° C. | 25° C. | 5° C. | 25° C. | 5° C. | 25° C. |
| (mg/mL) | | 5.08 | 5.06 | 4.99 | 4.97 | 4.89 | 4.97 |
| Recovery | 100.0 | 5° C. | 25° C. | 5° C. | 25° C. | 5° C. | 25° C. |
| (%) | | 100.0 | 99.6 | 98.3 | 97.8 | 97.8 | 97.8 |
| Impurity | N/D* | 5° C. | 25° C. | 5° C. | 25° C. | 5° C. | 25° C. |
| | | N/D | N/D | N/D | N/D | N/D | N/D |
| Osmolal- | 335 | 5° C. | 25° C. | 5° C. | 25° C. | 5° C. | 25° C. |
| ity | | N/D | N/D | N/D | N/D | N/D | N/D |
| (mOsm/ | | | | | | | |
| kg) | | | | | | | |
| Viscosity | 1.3 | 5° C. | 25° C. | 5° C. | 25° C. | 5° C. | 25° C. |
| (cP) | | N/D | N/D | N/D | N/D | N/D | N/D |

*N/D: not determined

TABLE 18
Stability measurements of potassium bumetanide salt

| Measuring | T0 | 1 week | | 2 weeks | | 4 weeks | |
|---|---|---|---|---|---|---|---|
| pH | 7.0 | 5° C. | 25° C. | 5° C. | 25° C. | 5° C. | 25° C. |
| | | 7.0 | 7.0 | 7.0 | 7.1 | 7.0 | 7.1 |
| Assay | 5.10 | 5° C. | 25° C. | 5° C. | 25° C. | 5° C. | 25° C. |
| (mg/mL) | | 5.18 | 5.10 | 4.97 | 4.96 | 5.00 | 5.03 |
| Recovery | 100.0 | 5° C. | 25° C. | 5° C. | 25° C. | 5° C. | 25° C. |
| (%) | | 101.5 | 100.0 | 97.4 | 97.2 | 97.9 | 98.6 |
| Impurity | N/D* | 5° C. | 25° C. | 5° C. | 25° C. | 5° C. | 25° C. |
| | | N/D | N/D | N/D | N/D | N/D | N/D |
| Osmolal- | 317 | 5° C. | 25° C. | 5° C. | 25° C. | 5° C. | 25° C. |
| ity | | N/D | N/D | N/D | N/D | N/D | N/D |
| (mOsm/ | | | | | | | |
| kg) | | | | | | | |
| Viscosity | 1.3 | 5° C. | 25° C. | 5° C. | 25° C. | 5° C. | 25° C. |
| (cP) | | N/D | N/D | N/D | N/D | N/D | N/D |

*N/D: not determined

For the arginine bumetanide salt, the initial pH was 7.0 and after four weeks was 7.0 and 7.1 for the sample stored at 5° C. and 25° C. respectively. The concentration of bumetanide in solution was initially found to be 5.07 mg/mL, and after four weeks, the concentrations were 4.89 and 4.97 mg/ml for the sample stored at 5° C. and 25° C. respectively. The percent assay recovery was initially 100% and after four weeks was 97.8% for both the sample stored at 5° C. and 25° C. Lastly, no impurity was detected initially or after four weeks for either sample as detected by HPLC.

These data demonstrate that the arginine bumetanide salt is stable for up to four weeks at either 5° C. or 25° C., as shown in Table 17. Additionally, the data demonstrate that the potassium bumetanide salt is stable for up to four weeks at either 5° C. or 25° C., as shown in Table 18.

### Example 7. Method for Formulation of the Pharmaceutical Formulation of the Arginine Salt of Bumetanide, Potassium Salt of Bumetanide, and Lysine Salt of Bumetanide

The pharmaceutical formulation of the arginine salt of bumetanide for subcutaneous administration to patients suffering from congestive heart failure was prepared by first dissolving 0.1 g of sodium carboxymethyl cellulose in 100 mL of deionized water and mixing to result in a 0.1% sodium carboxymethyl cellulose solution, which was filtered through a 0.2 μm nylon filter. Mannitol, benzyl alcohol, and 98.3% bumetanide was weighed out to 4 g, 0.5 g, and 0.5 g respectively and mixed. To this mixture, about 16 g of the 0.1% sodium carboxymethyl cellulose solution was added. To this solution, 0.45 g L-arginine was added and mixed using a vortex until the solution becomes clear. The pH of the solution is then adjusted using 1.0 N HCl to reach a final pH of 7.0. The formulation for the arginine salt of bumetanide at both a 1 g and 20 g scale were recorded in Table 19.

TABLE 19
Formulation for the arginine salt of bumetanide

| | F69 | |
|---|---|---|
| Excipient | mg/g | 20 g Batch amount (mg) |
| 98.3%, Bumetanide | 5.0 | 101.72 |
| Sodium CMC | 1.0 | 20 |
| (low viscosity) | | |
| Benzyl Alcohol | 5.0 | 100 |
| L-Arginine | 4.5 | 90 |
| Mannitol | 40.0 | 0.8 g |

Adjust pH with 1.0N HCl to 7.0

| | | |
|---|---|---|
| 0.1% CMC-Na | QS* | 20.0 g |

*QS: quantum sufficit

TABLE 20
Formulation composition and compounding table for 10
g batch of arginine salt of bumetanide, potassium
salt of bumetanide, and lysine salt of bumetanide

| | F42 | | F43 | | F44 | |
|---|---|---|---|---|---|---|
| | | 10 g Batch | | 10 g Batch | | 10 g Batch |
| | | amount | | amount | | amount |
| Excipient | mg/g | (g) | mg/g | (g) | Mg/g | (g) |
| Bumetanide | 5.0 | 0.05 | 5.0 | 0.05 | 5.0 | 0.05 |
| Sodium CMC | 1.0 | 0.01 | 1.0 | 0.01 | 1.0 | 0.01 |
| (low viscosity) | | | | | | |
| EDETATE | 1.0 | 0.01 | 1.0 | 0.01 | 1.0 | 0.01 |
| DISODIUM | | | | | | |
| dihydrate | | | | | | |
| L-Arginine | 4.5 | 0.045 | 0 | 0 | 0 | 0 |
| 1N KOH | 0 | 0 | 20 | 0.2 | 0 | 0 |
| L-Lysine | 0 | 0 | 0 | 0 | 4.0 | 40 |

Adjust pH with 1.0N HCl to 7.0

| | | | | | | |
|---|---|---|---|---|---|---|
| 0.1% CMC-Na | QS | 10.0 | QS | 10.0 | QS | 10.0 |
| Q.S. to | | | | | | |

TABLE 21
Characterization of Arginine and Potassium Salts of Bumetanide

| | Solubility of | | Osmolality | |
|---|---|---|---|---|
| Sample | Bumetanide (mg/mL) | pH | (mOsm/kg) | Viscosity |
| F42 | 4.92 | 6.7 | 279 | N/D |
| F43 | 5.04 | 7.0 | 268 | N/D |

A 10 g batch of the arginine salt of bumetanide (F42), potassium salt of bumetanide (F43), and lysine salt of bumetanide (F44) for subcutaneous administration to patients suffering from congestive heart failure were formulated according to Table 20. To a 250 mL beaker, 0.1 g of sodium CMC was added with stirring bar, and 100 mL of DI-water was added and mix well to get a 0.1% CMC-Na solution. Bumetanide and edetate disodium dihydrate were weighed out into a 15 mL tared falcon tube. To the tube, about 8 g of 0.1% CMC-Na solution was added and mixed. To this solution the amount of arginine, KOH, or lysine as described in Table 20 was added and mixed with a vortex until the solution became clear. Add appropriate amount of base, mix with vortex till get clear solution. At this point the pH was measured and adjusted to pH 7.0 if necessary. The 0.1% CMC-Na solution to a total weight of 10 g and mixed well. About 1 mL of solution was used to test osmolality. The osmolality was adjusted if need to about 290±10 mOsm/kg with either NaCl or mannitol. After the osmolality was adjusted, an assay and impurity analysis were performed, and the solution was measured for the viscosity and spray ability. The measure of solubility, pH, and osmolality are summarized in Table 21.

TABLE 22
Formulation composition and compounding table for arginine salt of
bumetanide (F69) and potassium salt of bumetanide (F70)

| | F69 | | F70 | |
|---|---|---|---|---|
| | | 100 g Batch | | 100 g Batch |
| Excipient | mg/g | amount (mg) | mg/g | amount (mg) |
| 98.3%, Bumetanide | 5.0 | 508.60 | 5.0 | 508.60 |
| Sodium CMC | 1.0 | 100 | 1.0 | 100 |
| (low viscosity) | | | | |
| Benzyl Alcohol | 5.0 | 500 | 5.0 | 500 |
| L-Arginine | 4.5 | 450 | 0 | 0 |
| 1N KOH | 0 | 0 | 20.0* | 2000 |
| Mannitol | 40.0 | 4000 | 40.0 | 4000 |

Adjust pH with 1.0N HCl to 7.0

| | | | | |
|---|---|---|---|---|
| 0.1% CMC-Na | QS | 100.0 g | QS | 100.0 g |

*0.78 mg/g of K+ equivalent

A 100 g batch of the arginine salt of bumetanide (F69) and potassium salt of bumetanide (F70) for subcutaneous administration to patients suffering from congestive heart failure were formulated according to Table 22. Into a 500 ml beaker, 0.3 g of sodium CMC was added with a stirring bar. 300 mL of DI-water was added and mixed well to get 0.1% CMC-Na solution. The mixture was then filtered through a 0.2 μm nylon filter.

For the arginine salt of bumetanide (F69), bumetanide, benzyl alcohol and mannitol were weighed out in a 125 mL tared Erlenmeyer flask with stir bar. To the flask, about 75 g of 0.1% CMC-Na solution was added and mixed. The appropriate amount of L-arginine was added, and the solution was mixed with vortex until the solution became clear. At this point, the pH was measured and adjusted to pH to 7.0 if necessary. An amount of 0.1% CMC-Na solution was added to bring the total weight to 100 g, and the solution was mixed well.

For the potassium salt of bumetanide (F70), sodium CMC was weighed out into a suitable beaker with stirring to which a volume of DI-water was added, and the solution was mixed well to generate a 0.1% CMC-Na solution. This solution was then filtered through a 0.2 μm nylon filter. Bumetanide, benzyl alcohol, and mannitol were then weighed out and added to a primary formulation container with stirring. To this container about ¾ ths of the 0.1% CMC-Na was added and mixed or stirred. To this solution, an appropriate amount of 1N KOH was titrated slowly in and mixed with stirring, until the solution became clear. The clear solution was allowed to equilibrate to until a steady pH was achieved at the pH range of 6.3 to 7.3. The pH was then measured and re-adjusted to 7.0 if it was necessary using 1N HCl. Enough of the 0.1% CMC-Na solution was added and mixed well to bring the solution to the appropriate weight. The osmolality was measured in order to check that is was in the specified range.

Both F69 and F70 were filtered through a 0.2 μm nylon filter. One vial of each formulation was used to perform an appearance, a pH, an assay/impurity, an osmolality, and an actuation dose test the results of which are summarized in Table 23.

TABLE 23
Characterization of F69 and F70

| | | | Assay | | Osmolality |
|---|---|---|---|---|---|
| ID | Appearance | pH | (mg/g) | Impurity | (mOsm/kg) |
| F69 | Clear colorless | 7.0 | 4.98 | ND | 333 |
| | solution | | | | |
| F70 | Clear colorless | 7.0 | 4.89 | ND | 321 |
| | solution | | | | |

ND: not detected

### Example 8. Characterization of Lead Formulations Over 5 Months

The arginine salt of bumetanide (F69), potassium salt of bumetanide (F70), bumetanide emulsion (FE-9), and bumetanide nano-emulsion (FNE-1) were examine for stability over a period of 5 months stored at 25° C. One sample of F70 was observed for 10 months with the bottle open to test stability. The appearance of each of the samples was recorded and summarized in Table 24.

TABLE 24
Appearance of formulations

| Sample | Lot | Appearance |
|---|---|---|
| F69_5M_25° C. | 338-2-6 | Clear colorless liquid; same as T0 |
| F70_5M_25° C. | 338-2-2 | Clear colorless liquid; same as T0 |
| F70_10M_25° C. | 338-2-2 | Clear colorless liquid; same as T0 |
| (opened bottle) | | |
| FE-9_5M_25° C. | 338-2-41 | opaque emulsion; same as T0 |
| FNE-1_5M_25° C. | 338-2-34 | Clear very light yellowish solution |
| | | with no particles; same as T0 |

The concentration for each of these formulations was measured using HPLC analysis after 5 months at 25° C. and one sample of F69 stored for 10 months. The resulting concentrations are summarized in Table 25. Additionally, the assay recovery over either 5 months or 10 months was calculated (Table 26).

TABLE 25
Concentration stability of formulations

| | | | | | Sample | QS to | HPLC | Calculated | Conc. |
|---|---|---|---|---|---|---|---|---|---|
| | | PA of | PA of | Average | Size | Volume | conc. | conc. | at T0 |
| Sample | Lot | Inj. 1 | Inj. 2 | PA | (g) | (mL) | (mg/mL) | (mg/g) | (mg/g) |
| F69_5 M | 338-2-6 | 12185.7 | 12170.2 | 12177.95 | 0.498 | 5 | 0.511 | 5.13 | 4.98 |
| F70_5 M | 338-2-2 | 12271.9 | 12194.7 | 12233.3 | 0.497 | 5 | 0.513 | 5.16 | 4.89 |
| FE-9_5 M | 338-2-41 | 11100.7 | 11055.2 | 11077.95 | 0.498 | 5 | 0.465 | 4.67 | 4.59 |
| FNE- | 338-2-34 | 11590.1 | 11619.3 | 11604.7 | 0.504 | 5 | 0.487 | 4.83 | 5.09 |
| 1_5 M | | | | | | | | | |
| F70_10 M | 338-2-2 | 12734.9 | 12707.4 | 12721.15 | 0.503 | 5 | 0.524 | 5.21 | 4.89 |

TABLE 26
Assay recovery vs T0

| | | | | Assay | % |
|---|---|---|---|---|---|
| | | | Assay | recovery | claim of |
| | | Assay | at 5M | at 5M or | 5 mg/mL |
| | | at T0 | or 10M | 10M vs | (Target |
| Sample | Lot | (mg/mL) | (mg/mL) | T0 (%) | 95-105%) |
| F69_5M_ | 338-2-6 | 4.98 | 5.13 | 103.01 | 103% |
| 25° C. | | | | | |
| F70_5M_ | 338-2-2 | 4.89 | 5.16 | 105.52 | 103% |
| 25° C. | | | | | |
| F70_10M_ | 338-2-2 | 4.89 | 5.21 | 106.53 | 104% |
| 25° C. | | | | | |
| FE-9_5M_ | 338-2-41 | 4.59 | 4.67 | 101.74 | 93.4% |
| 25° C. | | | | | |
| FNE-1_5M_ | 338-2-34 | 5.09 | 4.83 | 94.89 | 102% |
| 25° C. | | | | | |

The compositions of the potassium salt of bumetanide formulations are summarized in Table 27 along with each formulation's measured pH, appearance, osmolality, and assay recovery.

TABLE 27
Formulation composition and compounding potassium salt of bumetanide formulation
of F70 at 10, 15 and 16.5, 18 and 20 mg/mL bumetanide strengths

| | | % w/w | | | | | |
|---|---|---|---|---|---|---|---|
| Ingredient | Function | F79 | F80 | F81 | F82 | F83 | F84 |
| Bumetanide | API | 1 | 1.5 | 2 | 1 | 1.5 | 1.8 |
| USP | | | | | | | |
| Sodium CMC | Viscosity | 0.1 | 0.1 | 0.1 | 0.1 | 0.1 | 0.1 |
| (low viscosity) | control agent | | | | | | |
| USP | | | | | | | |
| Benzyl | Preservative | 0.5 | 0.5 | 0.5 | 0.5 | 0.5 | 0.5 |
| Alcohol NF | | | | | | | |
| 1N KOH ACS | pH modifier and | 4 | 6 | 8 | 4 | 6 | 8 |
| | adjust pH | | | | | | |
| Mannitol USP | Tonicity Agent | 4 | 4 | 4 | 2.5 | 2 | 2 |
| Water for | Medium | 90.4 | 87.9 | 85.4 | 91.9 | 89.9 | 87.6 |
| Injection USP | | | | | | | |
| 1N HCl, ACS, | pH adjustment | 6.8 | 6.9 | 7.5 | 7.2-7.4 | 7.2-7.4 | 7.2-7.5 |
| USP, EP | agent | | | | | | |

Assay, pH and Osmolality:

| ID | F79 | F80 | F81 |
|---|---|---|---|
| Assay After 1^{st} filtration, | 10.5 | 15.3 | 19.7 |
| mg/ml | | | |
| Assay After 3^{rd} filtration, | 10.1 | 14.9 | 19.6 |
| mg/ml | | | |
| pH (Before filtration) | 6.69 | 6.79 | 7.54 |
| pH (After 3^{rd} filtration) | 6.78 | 6.86 | 7.51 |
| Osmolality (After 1^{st} | 378 | 420 | 418 |
| filtration) (mOsm/Kg) | | | |

Assay, pH, and osmolality:

| ID | F82 | F83 | F84 |
|---|---|---|---|
| pH (Before filtration) | 7.35 | 7.34 | 7.47 |
| pH (After filtration and overnight @ | 7.02 | 7.22 | 7.25 |
| 2-8° C.) | | | |
| Osmolality (After filtration) (mOsm/Kg) | 286 | 325 | 352 |
| Assay After filtration (0.22 μm) (mg/ml) | 9.80 | 14.18 | 16.58 |

Particle size distribution analysis was performed to determine the average particle size for the FE-9 and FNE-1 bumetanide emulsions was measured using dynamic light scattering on a Malvern Panalytical Zetasizer. The average particle sizes for FE-9 and FNE-1 stored at room temperature over a period of 5 months are described in Table 28.

TABLE 28
Particle size analysis FE-9 and FNE-1 emulsions

| Sample Name | T (° C.) | Z-Ave (d · nm) | Pdl | D(v, 0.1) | D(v, 0.5) | D(v, 0.9) |
|---|---|---|---|---|---|---|
| FE-9_T0 | 25 | 112.0 | 0.173 | 41.4 | 74.8 | 227.0 |
| FE-9_5 M 25° C. | 25 | 129.9 | 0.106 | 61.5 | 103 | 254 |
| FNE-1_1 W | 25 | 11.3 | 0.609 | 1.0 | 1.4 | 2.2 |
| FNE-1_5 M_25° C. | 25 | 5.7 | 0.314 | 1.3 | 1.9 | 3.6 |

At 25° C. storage condition, the assay for F69 after 5 months showed a concentration of 5.13 mg/g, which was close to the TO assay, which was 4.98 mg/g. The assay for F70 after 5 months showed a concentration of 5.16 mg/g, which was close to the TO assay, which was 4.89 mg/g. After 10 months, the assay of F70 showed a concentration of 5.21 mg/g, which was close to the T0 assay, which is 4.89 mg/g. After 10 months the assay recovery for F70 was about 106.5% compared to T0. This may have been due to the fact that the sample was sampled from an opened container (previously used) and which may account for the loss of water over time when it was stored at 25° C., The % claim value after 10M at storage at RT was 104%. The assay of FE9 after 5 months measured a concentration of 4.67 mg/g, which was close to the TO assay, which was 4.59 mg/g. The assay of FNE-1 was measured as 4.83 mg/g, which was close to the TO assay, which was 5.09 mg/g. No impurities were observed in all 4 formulations when they were stored at 25° C. for 5 months. At 25° C. for 5 months, for F69, F70, FE-9, and FNE-1 formulations, the assay recoveries vs TO were all more than 94%. Particles, precipitation, and phase separation were not observed in all 4 formulations. These data indicate that all four formulations were stable for 5 months when they were stored at 25° C. Additionally, after 5 months, the particle size distributions of both FE-9 and FNE-1 were close to that of T0 or T=1W.

### Example 9. Subcutaneous Administration of an Arginine or Potassium Salt of Bumetanide to a Patient Suffering Congestive Heart Failure with Severe Edema Caused Inadequate Gastrointestinal Absorption

According to the methods described herein, a physician of skill in the art can treat a patient, such as a human patient, so as to reduce or alleviate symptoms of edema arising from congestive heart failure. To this end, a physician of skill in the art has the patient administer to themselves a potassium or arginine salt of bumetanide. The potassium or arginine salt of bumetanide is administered by the patient experiencing symptoms of congestive heart failure such as shortness of breath, fatigue or edema that not reduced with the patient's typical daily dosage of an oral diuretic. At this time, the patient administers the potassium or arginine salt of bumetanide subcutaneously. Typical dosages are administered based on body weight, and are in the range of about 0.5-10 mg of the potassium or arginine salt of bumetanide over a 12 hour period, and not exceeding 10 mg of the potassium or arginine salt of bumetanide over a 12 hour period without consulting a physician of skill in the art.

The potassium or arginine bumetanide salt is administered in one, two, three or four doses over a 4 hour period. Each dose consists of 100-150 μL of buffer containing the potassium or arginine bumetanide salt at a concentration of about 5-25 mg/mL. Each unit dose contains about 0.5-2.5 mg of the potassium or arginine bumetanide salt, such that four doses do not exceed 10 mg of the pharmaceutical composition. The potassium or arginine bumetanide salt is administered to the patient in an amount sufficient to treat the symptoms of congestive heart failure as is self-evaluated by the patient, including reduction of swelling, increase urine output and reduced shortness of breath.

### Example 10. Extended Release Formulation for Subcutaneous Administration

The methods and compositions described herein can provide a subcutaneously administered formulation that releases bumetanide to a patient for an extended period of time (e.g., 8 hours, 12 hours, 24 hours, 36 hours, 48 hours, 3 days, or 5 days of diuresis) as a long acting diuretic. The extended release formulation is used to treat a patient at risk for rehospitalization after treatment for congestive heart failure. The use of the long acting diuretic permits such patients to be treated on an outpatient basis, rather than undergo rehospitalization for edema-related symptoms. The extended release formulation is administered as a subcutaneous depot (e.g., a depot formed by an emulsion), which is released from the subcutaneous depot over an extended period of time.

The extended release formulation is formed from an aqueous emulsion of arginine bumetanide salt at a bumetanide concentration of about 7 to 15 mg/mL. Each dose consists of about 100-300 μL of the aqueous emulsion, and the dosing level may be determined by the body weight and health status of the patient. The emulsion is a mixture of formed from glycerol (ca. 2.25% weight), medium chain triglycerides (MCT) (ca. 10% weight) and lecithin (E-80) (ca. 1.2% weight).

The subcutaneous administration of the depot formulation allows the patient to benefit from a reliable diuretic effect over a period of days without complications. As a result of the extended release subcutaneous formulation of the arginine bumetanide emulsion, a patient at risk of rehospitalization following hospitalization for the treatment of congestive heart failure, receives treatment after discharge from the hospital, such that diuresis is achieved and symptoms such as shortness of breath, fatigue, and edema are relieved for an extended period of time.

### Example 11. Preparation of Bumetanide Salts

The arginine bumetanide salt was prepared by adding 1.00 g of bumetanide and 0.53 g arginine to a motor. To the arginine and bumetanide in the motor, about 15 mL of 200 proof ethanol diluted 50% with deionized water was added. A slurry was made by granulating manually with a pestle for about 30 minutes. The resulting slurry was transferred to a tared beaker. The slurry was then dried under vacuum at −20° C. overnight. After more than 24 hours drying under vacuum, a loose, fluffy white solid was obtained with a molar ratio of 1:1.1 bumetanide to arginine, as described in Table 30. All chemicals used in generating the arginine bumetanide salt are described in Table 29.

TABLE 29
Description of Chemicals Used in Arginine Bumetanide Salt Preparation

| Description | Grade | Supplier | Lot |
|---|---|---|---|
| Bumetanide | N/A | CAYMAN CHEMICAL | 0472995.21 |
| Arginine | USP | Spectrum | 1EG0013 |
| Ethanol 200 proof | USP | Decon Lab, Inc. | 256915 |
| DI-H2O | N/A | Latitude | N/A |

TABLE 30
Ratio of Bumetanide to Arginine

| Description | Weight (g) | MW (g/mol) | Molar (M) | Molar ratio |
|---|---|---|---|---|
| Bumetanide | 1.00 | 364.417 | 0.00192 | 1:1.1 |
| Arginine | 0.53 | 174.200 | 0.00213 | |

The potassium bumetanide salt was prepared by adding 1.0 g of bumetanide to a 20 mL glass vial. To the glass vial, 5 mL of a 200 proof ethanol solution diluted by 50% with deionized water was added and vortexed to mix for about 1 minute. To the wet bumetanide, 3.0 mL of 1 N KOH solution was added and mixed with a vortex or a spatula for about 10 minutes resulting in a white slurry. The mixture was frozen for about 1 hour at −20° C. The mixture was then dried under vacuum overnight at −20° C. Once dried, the white, fluffy soft solid potassium bumetanide, having a molar ratio of 1:1.1 bumetanide to potassium as described in Table 32, was transferred to a clean glass vial. The potassium bumetanide salt was reserved at 2-8° C. for formulation preparation. All chemicals used in generating the potassium bumetanide salt are described in Table 31.

TABLE 31
Description of Chemicals Used in Potassium Bumetanide Salt Preparation

| Description | Grade | Supplier | Lot |
|---|---|---|---|
| Bumetanide | N/A | CAYMAN CHEMICAL | 0472995.21 |
| 1.0N KOH | USP | Spectrum | 1EG0013 |
| Ethanol 200 proof | USP | Decon Lab., Inc | 256915 |
| DI-H2O | N/A | Latitude | N/A |

TABLE 32
Ratio of Potassium to Arginine

| Description | Weight (g) | MW | Molar | Molar ratio |
|---|---|---|---|---|
| Bumetanide | 1.0 | 364.417 | 0.0027 | 1:1.11 |
| 1N KOH | 3.0 | 56.11 | 0.0030 | |

### Example 12. Characterization of Subcutaneous Formulations of Potassium and Arginine Salts of Bumetanide

About 3.7 g of sterile water for injection (SWFI) was weighed and added to two tared 15 mL centrifuge tubes. One tube was labeled as FS-1 and the other as FS-3. Bumetanide was added to each tube as described by Table 33. To each solution, the appropriate base was added (arginine to FS-1 and KOH to FS-3). The solutions were then mixed to dissolve the bumetanide. If the bumetanide was not dissolved, more base was slowly added, and the amount added was recorded. To each solution, the benzyl alcohol and mannitol were added, and was brought to 5.0 g with SWFI. The solutions were vortexed to mix and the initial pH was measured. 2.5 g of FS-1 was separated into 15 mL falcon tube and was labelled as FS-2. 2.5 g of FS-3 was separated into a 15 mL falcon tube and labelled as FS-4. The pH was adjusted for each formulation as described in Table 33 using 1N HCl, and the final pH was recorded.

TABLE 33
Formulations of bumetanide for characterization

| Ingredient | Composition | | | | | | | | |
|---|---|---|---|---|---|---|---|---|---|
| Description | mg/g | FS-1 | | FS-2 | | FS-3 | | FS-4 | |
| Bumetanide | 10 | 50.86 | mg | 50.86 | mg | 50.86 | mg | 50.86 | mg |
| Benzyl | 5 | 25.0 | mg | 25.0 | mg | 25.0 | mg | 25.0 | mg |
| Alcohol | | | | | | | | | |
| L-Arginine | Add slowly | 45.0 | mg | 45.0 | mg | 0.0 | | 0.0 | |
| | until API | | | | | | | | |
| | dissolves | | | | | | | | |
| 1N KOH | Add slowly | 0.0 | | 0.0 | | 190 | μL | 190 | μL |
| | until API | | | | | | | | |
| | dissolves | | | | | | | | |
| Mannitol | 40 | 0.2 | g | 0.2 | g | 0.2 | g | 0.2 | g |
| SWFI | QS to | 5.0 | g | 5.0 | g | 5.0 | g | 5.0 | g |

| | | | | | |
|---|---|---|---|---|---|
| (Sterile water | | | | | |
| for injection) | | | | | |
| Target pH | — | 7.4 | 6.5 | 7.4 | 6.5 |

Adjust pH to target with 1N HCl if needed

TABLE 34
Characterization of bumetanide formulations
Final pH and Appearance:

| Sample ID | Base added | Final pH | Appearance |
|---|---|---|---|
| FS-1 | Arginine | 7.53 | Clear at first, but white precipitates crash out over |
| | | | time |
| FS-2 | Arginine | 6.75 | Clear at first, but white precipitates crash out over |
| | | | time |
| FS-3 | KOH | 7.34 | Clear and transparent |
| FS-4 | KOH | 6.73 | Clear and transparent |

Osmolality Results:

| Sample ID | Base added | Final pH | Osmolality (mOsm/kg) |
|---|---|---|---|
| 290 mOsm | — | — | 288 |
| Standard | | | |
| FS-3 | KOH | 7.34 | 365 |
| FS-4 | KOH | 6.73 | 333 |

HPLC Results

| | | | | HPLC | Sample | Total | Cal. |
|---|---|---|---|---|---|---|---|
| | | | | conc. | size | Weight | Con. |
| Sample ID | Inj 1 | Inj 2 | Ave. PA | (mg/mL) | (g) | (mL) | (mg/g) |
| FS-3 | 13524 | 12618 | 13071 | 0.5439 | 0.26756 | 5 | 10.2 |
| FS-4 | 13723 | 13681 | 13702 | 0.5702 | 0.29212 | 5 | 9.8 |

The various formulations were characterized in terms of appearance, pH, osmolality, and bumetanide concentration, as is summarized in Table 34. The bumetanide concentration was measured using an HPLC assay using the conditions described in Table 35. The HPLC assay was performed by preparing 2 L of methanol:distilled water:tetrahydrofuran:glacial acetic acid in a ratio of %):45:5:2 (v/v/v/v) and filtering the solution through a 0.8 μM nylon membrane filter. A bumetanide standard having a concentration of 0.125 mg/mL was prepared by weighing out 12.5 mg of bumetanide into a 100 mL volumetric flask and dissolving in 100 mL of stock diluent, which was made by adding 10 mL of tetrahydrofuran to a 100 mL volumetric flask along with 4 mL of glacial acetic acid and bringing to 100 mL with methanol. The sample solution was prepared by weighing out 125 mg of sample into a 10 mL volumetric flask and bringing to 10 mL with distilled water.

TABLE 35
HPLC assay conditions

| HPLC System | Agilent 1200 | |
|---|---|---|
| Analytical Column | Waters μBondapak C18 10 μm Irregular | |
| | Silica 3.9 × 300 mm | |
| | P/N: WAT027324(#527) | |
| Mobile Phase 1 (MP1) | MeOH: DI H₂O:THF:GAA (50:45:5:2, v/v/v/v) | |
| Flow Rate | 1 | mL/min |
| Detection Wavelength | 254 | nm |
| Column Temperature | 40° | C. |
| Injection Volume | 20 | μL |
| Run Time | 35 | minutes |
| Sample diluent | DI-water | |
| Nominal Concentration | 0.125 | mg/ml (API) |

The FS-3 and FS-4 formulations with bumetanide potassium salt were clear and transparent at pH 7.4 and 6.7. The HPLC results showed that the assay was within the expected targeted range for bumetanide (10 mg/mL) at both pH values. For FS-3, the osmolality was slightly higher than FS-4. The FS-1 and FS-2 formulations with bumetanide arginine salt were clear at first, but white precipitates crashed out over time for both pH values using these conditions.

Mannitol was adjusted in the potassium bumetanide formulations from 4% to 3% (w/w) in the formulation to generate an osmolality in the range of 280-360 mOSm/kg. The resulting osmolality is described in Table 36.

TABLE 36
Osmolality of potassium bumetanide formulations
with adjusted mannitol concentration

| Sample ID | Base added | Final pH | Osmolality (mOsm) |
|---|---|---|---|
| 290 mOsm Standard | — | — | 290 |
| FS-3 w/3% mannitol | KOH | 7.55 | 292 |
| FS-4 w/3% mannitol | KOH | 6.72 | 312 |

### Example 13. Formulations of Bumetanide Salts for Administration to a Subject

To prepare the bumetanide formulations for administration, about 162.8 g of SWFI was weighed into two tared 250 ml Nalgene bottles with a stir bar. One was labelled as FS-1 and the other as FS-3. To the bottles, bumetanide was added as is described in Tables 37 and 38. The arginine base (FS-5) or KOH base (FS-3) were then added to the solution and mixed to dissolve the bumetanide. If the bumetanide was not dissolved, more base was slowly added and recorded. To the solutions, benzyl alcohol and mannitol were added and brought to 5.0 g with SWFI. The solutions were vortexed to mix and the initial pH was measured. 110 g of FS-1 was split into a separate 100 ml Nalgene bottle and labeled as FS-2. 110 g of FS-3 was split into a separate 100 ml Nalgene bottle and labeled as FS-4. For FS-2 and FS-4, the pH was adjusted to ˜6.7. For FS-1 and FS-3, the pH was adjusted to ˜7.4. The solutions were filtered through a 0.22 μm filter in the biosafety hood. The appearance, bumetanide concentration, impurities, pH, and osmolality were assessed.

TABLE 37
Formulation of bumetanide for administration

| | Composition | FS-6 | FS-5 | FS-3 | FS-4 |
|---|---|---|---|---|---|
| | Description | | | | |
| | | Arginine, pH | Arginine, | | KOH, pH |
| Ingredient | mg/g | 7.4 | pH 6.7 | KOH, pH 7.4 | 6.7 |
| 99.8%, Bumetanide | 10 | 2204.4 mg | 2204.4 mg | 2204.4 mg | 2204.4 mg |
| USP | | | | | |
| Benzyl Alcohol | 5 | 1100 mg | 1100 mg | 1100 mg | 1100 mg |
| L-Arginine* | See columns | 1760 mg | 1760 mg | 0.0 | 0.0 |
| | 3 and 4 | | | | |
| 1N KOH** | See columns | 0.0 | 0.0 | 8.0 mL | 8.0 mL |
| | 5 and 6 | | | | |
| Mannitol | 30 | 6.6 g | 6.6 g | 6.6 g | 6.6 g |
| SWFI | QS | To 220 g | To 220 g | To 220 g | To 220 g |

*Expected arginine range to add: 1760 mg to 2200 mg (FS-1 and FS-2 are not continued because a lot of precipitate observed in the formulation)
**Expected KOH range to add: 7.9 mL to 8.8 mL

The Arginine bumetanide formulations for subcutaneous administration, FS-5 and FS-6, were made by taring two 15 mL falcon tubes and labelling one as FS-5 and the other as FS-6. To each tube, about 8 g of sterile water for injection (SWFI) was added and mixed well until the arginine was completely dissolved. To this solution, benzyl alcohol, bumetanide, and mannitol was added as described in Table 38. The solution was brought to 10 g with SWFI, vortexed to mix, and an initial pH was measured. The pH was adjusted to pH 7.0 for formulation FS-5 and to pH 7.2 for FS-6 with glacial acetic acid and then mixed with a vortex for 5-10 minutes. The solutions were left at room temperature overnight and protected from light. The solutions were then filtered through 0.22 μm filter. The solutions were distributed as 1.0 mL aliquots into glass vials which were crimp sealed. Each formulation was assessed for concentration, pH, and osmolality.

The intranasal formulation of bumetanide (F82) was generated according to Table 39.

TABLE 38
Subcutaneous formulations of FS-3. FS-4, FS-5 and FS-6

| Ingredient | FS-3 (%, w/w) | FS-4 (%, w/w) | FS-5 (%, w/w) | FS-6 (%, w/w) |
|---|---|---|---|---|
| Bumetanide | 1.0 | 1.0 | 1.0 | 1.0 |
| Arginine | — | — | 0.6 | 0.6 |
| 1N KOH | 2.0 | 2.0 | — | — |
| Benzyl Alcohol | 0.5 | 0.5 | 0.5 | 0.5 |
| Mannitol | 3.0 | 3.0 | 3.30 | 3.53 |
| SWFI | 93.5 | 93.5 | 94.9 | 94.9 |
| Adjust pH with 0.1N HCl | pH = 7.4 | pH = 6.7 | — | — |
| Adjust pH with glacial acetic acid | — | — | pH = 6.7 | pH = 7.2 |
| Test data: | | | | |
| pH | 7.2 | 6.8 | 6.7 | 7.2 |
| Assay (Concentration) | 10.1 mg/ml | 10.1 mg/mL | 9.7 mg/mL | 10.0 mg/ml |
| Osmolaity | 285 mOsm/kg | 282 mOsm/kg | 307 mOsm/kg | 311 mOsm/kg |

TABLE 39
Intranasal formulation of F82

| Ingredient | F82 (%, w/w) |
|---|---|
| Bumetanide | 1.0 |
| Sodium CMC (low viscosity) | 0.1 |
| Benzyl Alcohol | 0.5 |
| 1.0N KOH | 4.0 |
| Mannitol | 3.1 |
| Water for Injection USP | 91.3 |
| Adjust pH with 1.0N HCl | pH = 6.6 |
| Test data: | |
| pH | 6.6 |
| Assay | 1.01% w/w |
| Osmolaity | 302 mOsm/kg |

### Example 14. Stability of Formulations of Bumetanide Salts

### Stability of Potassium Salt Formulations

The FS-3 and FS-4 formulations were tested for stability for 3 months at 25° C. and 40° C. The formulations were filled into type 1 clear glass vials that were stoppered and crimp-sealed. The vials were then placed into 25° C. and 40° C. storage conditions (protected from light) for stability testing. Each sample was pulled and tested according to Table 39 below. The results of the stability experiments are summarized in Tables 40-44.

TABLE 39
Stability Time Points
Bumetanide SubQ formulation Stability Time Points/Condition:

| Storage Condition | T0 | 0.5M | 1M | 2M | 3M |
|---|---|---|---|---|---|
| 25° C. | A | B | B | B | B |
| 40° C. | | B | B | B | B |

Panel A (TO only): Was studied for appearance, pH, osmolality, injectability (27G-30G) as per method in the appendix, density/viscosity, HPLC assay/Impurities, particulate matter by USP <788> method (visual).
Panel B (0.5M, 1M, 2M, 3M timepoints): Was studied for appearance, pH, HPLC assay/impurities, viscosity, particulate matter by USP <788> method (visual).

TABLE 40
Summary of FS-3 formulation at 25° C.
FS-3 @ 25° C. Stability Results

| Time Point | T0 | 0.5 M | 1 M | 2 M | 3 M | Comments |
|---|---|---|---|---|---|---|
| Appearance | Clear and | Clear with | Clear with | Clear with | Clear with | N/A |
| | transparent | a few | a few | a few | a few | |
| | solution | needle-like | needle-like | needle-like | needle-like | |
| | | precipitates | precipitates | precipitates | precipitates | |
| Assay | 9.78 mg/mL | 100.51% | 101.94% | 99.46% | 99.49% | N/A |
| | | 9.83 | 9.97 | 9.73 | 9.73 | N/A |
| Imp. A (RRT = 0.59 mins) | <0.05% | <0.05% | <0.05% | <0.05% | <0.05% | N/A |
| Imp. B (RRT = 0.42 mins) | 0.05% | <0.05% | <0.05% | <0.05% | <0.05% | N/A |
| Imp. C (RRT = 3.12 mins) | <0.05% | <0.05% | <0.05% | N/D | N/D | N/A |
| Imp. D (RRT = 2.50 mins) | <0.05% | <0.05% | <0.05% | N/D | N/D | N/A |
| Total Impurities (%) | 0.07% | 0.07% | 0.01% | 0.02% | 0.01% | N/A |
| pH | 7.5 | 7.7 | 7.5 | 7.3 | 7.9 | N/A |
| Osmolality (mOsm/kg) | 297 | N/D | N/D | N/D | N/D | N/A |
| Injectability and | Pass | N/D | N/D | N/D | N/D | 27G to |
| Syringeability | | | | | | 30G |
| Density (g/mL) at RT | 0.99 | N/D | N/D | N/D | N/D | N/A |
| Viscosity (cP) | 1.04 | 1.04 | 0.89 | 1.02 | 1.02 | N/A |
| Particulate Matter | 30 | 216 | 364 | 58 | 82 | 10 μm ≤ 6000 |
| | 0 | 82 | 44 | 4 | 10 | 25 μm ≤ 600 |

N/D: not detected

TABLE 41
Summary of FS-3 formulation at 40° C.
FS-3 @ 40° C. Stability Results

| Time Point | T0 | 0.5 M | 1 M | 2 M | 3 M | Comments |
|---|---|---|---|---|---|---|
| Appearance | Clear and | Clear and | Clear and | Clear and | Clear and | N/A |
| | transparent | transparent | transparent | transparent | transparent | |
| | solution | solution | solution | solution | solution | |
| Assay | 9.78 mg/mL | 100.51% | 101.96% | 100.07% | 99.90% | N/A |
| | | 9.83 | 9.97 | 9.79 | 9.77 | N/A |
| Imp. A (RRT = 0.59 mins) | <0.05% | <0.05% | <0.05% | <0.05% | <0.05% | N/A |
| Imp. B (RRT = 0.42 mins) | 0.05% | <0.05% | <0.05% | <0.05% | <0.05% | N/A |
| Imp. C (RRT = 3.12 mins) | <0.05% | <0.05% | <0.05% | N/D | N/D | N/A |
| Imp. D (RRT = 2.50 mins) | <0.05% | <0.05% | <0.05% | N/D | N/D | N/A |
| Total Impurities (%) | 0.07% | 0.07% | 0.02% | 0.02% | 0.01% | N/A |
| pH | 7.5 | 7.3 | 7.7 | 7.4 | 7.6 | N/A |
| Osmolality (mOsm/kg) | 297 | N/D | N/D | N/D | N/D | N/A |
| Injectability and | Pass | N/D | N/D | N/D | N/D | 27G to |
| Syringeability | | | | | | 30G |
| Density (g/mL) at RT | 0.99 | N/D | N/D | N/D | N/D | N/A |
| Viscosity (cP) | 1.04 | 1.05 | 1.03 | 1.04 | 1.02 | N/A |
| Particulate Matter | 30 | 326 | 876 | 138 | 158 | 10 μm ≤ 6000 |
| | 0 | 150 | 234 | 30 | 10 | 25 μm ≤ 600 |

N/D: not detected

TABLE 42
Summary of FS-4 formulation at 25° C.
FS-4 @ 25° C. Stability Results

| Time Point | T0 | 0.5 M | 1 M | 2 M | 3 M | Comments |
|---|---|---|---|---|---|---|
| Appearance | Clear and | Clear with | Clear with | Clear with | Clear with | N/A |
| | transparent | a few | a few | a few | a few | |
| | solution | needle-like | needle-like | needle-like | needle-like | |
| | | precipitates | precipitates | precipitates | precipitates | |
| Assay | 9.73 mg/mL | 100.18% | 102.02% | 99.37% | 99.48% | N/A |
| | | 9.75 | 9.93 | 9.67 | 9.68 | N/A |
| Imp. A (RRT = 0.59 mins) | <0.05% | <0.05% | <0.05% | <0.05% | <0.05% | N/A |
| Imp. B (RRT = 0.42 mins) | 0.05% | <0.05% | <0.05% | <0.05% | <0.05% | N/A |
| Imp. C (RRT = 3.12 mins) | <0.05% | <0.05% | <0.05% | N/D | N/D | N/A |
| Imp. D (RRT = 2.50 mins) | <0.05% | <0.05% | <0.05% | N/D | N/D | N/A |
| Total Impurities (%) | 0.07% | 0.07% | 0.01% | 0.02% | 0.01% | N/A |
| pH | 6.9 | 7.2 | 7.3 | 7.1 | 7.8 | N/A |
| Osmolality (mOsm/kg) | 294 | N/D | N/D | N/D | N/D | N/A |
| Injectability and | Pass | N/D | N/D | N/D | N/D | 27G to |
| Syringeability | | | | | | 30G |
| Density (g/mL) at RT | 0.99 | N/D | N/D | N/D | N/D | N/A |
| Viscosity (cP) | 1.04 | 1.04 | 1.02 | 1.04 | 1.02 | N/A |
| Particulate Matter | 22 | 36 | 20 | 334 | 88 | 10 μm ≤ 6000 |
| | 6 | 0 | 4 | 92 | 6 | 25 μm ≤ 600 |

N/D: not detected

TABLE 43
Summary of FS-4 formulation at 40° C.
FS-4 @ 40° C. Stability Results

| Time Point | T0 | 0.5 M | 1 M | 2 M | 3 M | Comments |
|---|---|---|---|---|---|---|
| Appearance | Clear and | Clear and | Clear and | Clear and | Clear and | N/A |
| | transparent | transparent | transparent | transparent | transparent | |
| | solution | solution | solution | solution | solution | |
| Assay | 9.73 mg/mL | 99.92% | 101.71% | 100.27% | 100.26% | N/A |
| | | 9.72 | 9.90 | 9.76 | 9.76 | N/A |
| Imp. A (RRT = 0.59 mins) | <0.05% | <0.05% | <0.05% | <0.05% | <0.05% | N/A |
| Imp. B (RRT = 0.42 mins) | 0.05% | <0.05% | <0.05% | <0.05% | <0.05% | N/A |
| Imp. C (RRT = 3.12 mins) | <0.05% | <0.05% | <0.05% | N/D | N/D | N/A |
| Imp. D (RRT = 2.50 mins) | <0.05% | <0.05% | <0.05% | N/D | N/D | N/A |
| Total Impurities (%) | 0.07% | 0.07% | 0.01% | 0.02% | 0.01% | N/A |
| ph | 6.9 | 7.2 | 7.1 | 7.0 | 7.4 | N/A |
| Osmolality (mOsm/kg) | 294 | N/D | N/D | N/D | N/D | N/A |
| Injectability and | Pass | N/D | N/D | N/D | N/D | 27G to |
| Syringeability | | | | | | 30G |
| Density (g/mL) at RT | 0.99 | N/D | N/D | N/D | N/D | N/A |
| Viscosity (cP) | 1.04 | 1.04 | 0.98 | 1.04 | 1.02 | N/A |
| Particulate Matter | 22 | 36 | 242 | 88 | 278 | 10 μm ≤ 6000 |
| | 6 | 0 | 56 | 2 | 28 | 25 μm ≤ 600 |

N/D: not detected. For PM testing, as per USP, 10 μm ≤ 6000; 25 μm ≤ 600

TABLE 44
Appearance of FS-3 and FS-4 Formulations at Two Months

| Sample ID | Appearance |
|---|---|
| FS-3_25° C._2M | Clear with a few needle-like precipitation |
| FS-3_40° C._2M | Clear and transparent solution |
| FS-4_25° C._2M | Clear with a few needle-like precipitation |
| FS-4_40° C._2M | Clear and transparent solution |

### Stability of Arginine Salt Formulations

The FS-5 and FS-6 formulations were tested for stability for 9 months at 25° C. and 40° C. The formulations were filled into type 1 clear glass vials that were stoppered and crimp-sealed. The vials were then placed into 25° C. and 40° C. storage conditions (protected from light) for stability testing. Each sample was pulled and tested according to Table 45 below. The results of the stability experiments are summarized in Tables 46-50.

TABLE 45
Stability Time Points
Bumetanide SubQ formulation Stability Time Points/Condition:

| Storage Condition | T0 | 0.5M | 1M | 2M | 3M |
|---|---|---|---|---|---|
| 25° C. | A | B | B | B | B |
| 40° C. | | B | B | B | B |

Panel A (TO only): Was studied for appearance, pH, osmolality, injectability (27G-30G) as per method in the appendix, density/viscosity, HPLC assay/impurities, and particulate matter by USP <788> method (visual).
Panel B (0.5M, 1M, 2M, 3M timepoints): Was studied for appearance, pH, HPLC assay/impurities, viscosity, and particulate matter by USP <788> method (visual).

TABLE 46
Summary of FS-5 formulation at 25° C.
FS-5 @ 25° C. Stability Results

| Time Point | T0 | 0.5 M | 1 M | 2 M | 3 M | 9 M | Comments |
|---|---|---|---|---|---|---|---|
| Appearance | Clear and | Clear and | Clear and | Clear and | Clear and | Clear and | N/A |
| | transparent | transparent | transparent | transparent | transparent | transparent | |
| | solution | solution | solution | solution | solution | solution | |
| Assay | 10.12 mg/mL | 99.15% | 98.22% | 99.79% | 97.60% | 98.05% | N/A |
| | | 10.04 mg/mL | 9.94 mg/mL | 10.10 mg/mL | 9.79 mg/mL | 9.92 mg/mL | N/A |
| Imp. A (RRT = | <0.05% | <0.05% | <0.05% | <0.05% | <0.05% | <0.05% | N/A |
| 0.59 mins) | | | | | | | |
| Imp. B (RRT = | <0.05% | <0.05% | <0.05% | <0.05% | <0.05% | <0.05% | N/A |
| 0.42 mins) | | | | | | | |
| Imp. C (RRT = | <0.05% | <0.05% | <0.05% | <0.05% | <0.05% | N/D | N/A |
| 3.12 mins) | | | | | | | |
| Imp. D (RRT = | <0.05% | <0.05% | <0.05% | <0.05% | <0.05% | N/D | N/A |
| 2.50 mins) | | | | | | | |
| Total Impurities | 0.05% | 0.05% | 0.05% | 0.02% | 0.03% | 0.04% | N/A |
| (%) | | | | | | | |
| pH | 6.8 | 7.1 | 6.9 | 7.2 | 7.2 | 7.3 | N/A |
| Osmolality | 304 | N/D | N/D | N/D | N/D | N/D | N/A |
| (mOsm/kg) | | | | | | | |
| Injectability and | Passes | N/D | N/D | N/D | N/D | N/D | 27G to |
| Syringeability | | | | | | | 30G |
| Density (g/mL) at | 1.01 | N/D | N/D | N/D | N/D | N/D | N/A |
| RT | | | | | | | |
| Viscosity (cP) | 1.05 | N/D | N/D | 0.91 | 1.08 | 1.07 | N/A |
| Particulate | 1050 | Passes | Passes | 626 | 422 | N/D | 10 μm ≤ |
| Matter | | visual | visual | | | | 6000 |
| | 18 | Passes | Passes | 190 | 26 | N/D | 25 μm ≤ |
| | | visual | visual | | | | 600 |

ND: not done

TABLE 47
Summary of FS-5 formulation at 40° C.

| | | FS-5 @ 40° C. Stability Results | | | | | |
|---|---|---|---|---|---|---|---|
| Time Point | T0 | 0.5 M | 1 M | 2 M | 3 M | 9 M | Comments |
| Appearance | Clear and | Clear and | Clear and | Clear and | Clear and | Clear and | N/A |
| | transparent | transparent | transparent | transparent | transparent | transparent | |
| | solution | solution | solution | solution | solution | solution | |
| Assay | 10.12 mg/mL | 99.45% | 98.47% | 100.61% | 98.07% | 99.30% | N/A |
| | | 10.07 mg/mL | 9.97 mg/mL | 10.19 mg/mL | 9.84 mg/mL | 10.05 mg/mL | N/A |
| Imp. A (RRT = | <0.05% | <0.05% | <0.05% | <0.05% | <0.05% | <0.05% | N/A |
| 0.59 mins) | | | | | | | |
| Imp. B (RRT = | <0.05% | <0.05% | <0.05% | <0.05% | <0.05% | 0.11% | N/A |
| 0.42 mins) | | | | | | | |
| Imp. C (RRT = | <0.05% | <0.05% | <0.05% | <0.05% | <0.05% | N/D | N/A |
| 3.12 mins) | | | | | | | |
| Imp. D (RRT = | <0.05% | <0.05% | <0.05% | <0.05% | <0.05% | N/D | N/A |
| 2.50 mins) | | | | | | | |
| Total | 0.05% | 0.04% | 0.04% | 0.04% | 0.05% | 0.15% | N/A |
| Impurities | | | | | | | |
| (%) | | | | | | | |
| pH | 6.8 | 7.0 | 7.0 | 7.2 | 7.2 | 7.1 | N/A |
| Osmolality | 304 | N/D | N/D | N/D | N/D | N/D | N/A |
| (mOsm/kg) | | | | | | | |
| Injectability | Passes | N/D | N/D | N/D | N/D | N/D | 27G to |
| and | | | | | | | 30G |
| Syringeability | | | | | | | |
| Density | 1.01 | N/D | N/D | N/D | N/D | N/D | N/A |
| (g/mL) at RT | | | | | | | |
| Viscosity | 1.05 | N/D | N/D | 1.00 | 1.07 | 1.08 | N/A |
| (cP) | | | | | | | |
| Particulate | 1050 | Passes | Passes | 152 | 568 | N/D | 10 μm ≤ |
| Matter | | visual | visual | | | | 6000 |
| | 18 | Passes | Passes | 6 | 108 | N/D | 25 μm ≤ |
| | | visual | visual | | | | 600 |

ND: not done

TABLE 48
Summary of FS-6 formulation at 25° C.

| | | FS-6 @ 25° C. Stability Results | | | | | |
|---|---|---|---|---|---|---|---|
| Time Point | T0 | 0.5 M | 1 M | 2 M | 3 M | 9 M | Comments |
| Appearance | Clear and | Clear and | Clear and | Clear and | Clear and | Clear and | N/A |
| | transparent | transparent | transparent | transparent | transparent | transparent | |
| | solution | solution | solution | solution | solution | solution | |
| Assay | 10.01 mg/mL | 99.84% | 100.19% | 102.27% | 100.23% | 99.91% | N/A |
| | | 9.99 mg/mL | 10.02 mg/mL | 10.23 mg/mL | 99.93 mg/mL | 10.00 mg/mL | N/A |
| Imp. A (RRT = | <0.05% | <0.05% | <0.05% | <0.05% | <0.05% | <0.05% | N/A |
| 0.59 mins) | | | | | | | |
| Imp. B (RRT = | <0.05% | <0.05% | <0.05% | <0.05% | <0.05% | <0.05% | N/A |
| 0.42 mins) | | | | | | | |
| Imp. C (RRT = | <0.05% | <0.05% | <0.05% | <0.05% | <0.05% | N/D | N/A |
| 3.12 mins) | | | | | | | |
| Imp. D (RRT = | <0.05% | <0.05% | <0.05% | <0.05% | <0.05% | N/D | N/A |
| 2.50 mins) | | | | | | | |
| Total | 0.05% | 0.04% | 0.04% | 0.01% | 0.02% | 0.04% | N/A |
| Impurities | | | | | | | |
| (%) | | | | | | | |
| pH | 7.2 | 7.3 | 7.3 | 7.5 | 7.5 | 7.6 | N/A |
| Osmolality | 315 | N/D | N/D | N/D | N/D | N/D | N/A |
| (mOsm/kg) | | | | | | | |
| Injectability | Passes | N/D | N/D | N/D | N/D | N/D | 27G to |
| and | | | | | | | 30G |
| Syringeability | | | | | | | |
| Density | 1.01 | N/D | N/D | N/D | N/D | N/D | N/A |
| (g/mL) at RT | | | | | | | |
| Viscosity | 1.05 | N/D | N/D | 1.04 | 1.07 | 1.08 | N/A |
| (cP) | | | | | | | |
| Particulate | 226 | Passes | Passes | 450 | 458 | N/D | 10 μm ≤ |
| Matter | | visual | visual | | | | 6000 |
| | 10 | Passes | Passes | 136 | 94 | N/D | 25 μm ≤ |
| | | visual | visual | | | | 600 |

ND: not done

TABLE 49
Summary of FS-6 formulation at 40° C.
FS-6 @ 40° C. Stability Results

| Time Point | T0 | 0.5 M | 1 M | 2 M | 3 M | 9 M | Comments |
|---|---|---|---|---|---|---|---|
| Appearance | Clear and | Clear and | Clear and | Clear and | Clear and | Clear and | N/A |
| | transparent | transparent | transparent | transparent | transparent | transparent | |
| | solution | solution | solution | solution | solution | solution | |
| Assay | 10.01 mg/mL | 100.32% | 99.33% | 101.07% | 99.64% | 100.94% | N/A |
| | | 10.04 mg/mL | 9.94 mg/mL | 10.11 mg/mL | 9.87 mg/mL | 10.10 mg/mL | N/A |
| Imp. A (RRT = | <0.05% | <0.05% | <0.05% | <0.05% | <0.05% | 0.07% | N/A |
| 0.59 mins) | | | | | | | |
| Imp. B (RRT = | <0.05% | <0.05% | <0.05% | <0.05% | <0.05% | 0.14% | N/A |
| 0.42 mins) | | | | | | | |
| Imp. C (RRT = | <0.05% | <0.05% | <0.05% | <0.05% | <0.05% | N/D | N/A |
| 3.12 mins) | | | | | | | |
| Imp. D (RRT = | <0.05% | <0.05% | <0.05% | <0.05% | <0.05% | N/D | N/A |
| 2.50 mins) | | | | | | | |
| Total | 0.05% | 0.04% | 0.05% | 0.03% | 0.05% | 0.20% | N/A |
| Impurities | | | | | | | |
| (%) | | | | | | | |
| pH | 7.2 | 7.4 | 7.4 | 7.5 | 7.5 | 7.5 | N/A |
| Osmolality | 315 | N/D | N/D | N/D | N/D | N/D | N/A |
| (mOsm/kg) | | | | | | | |
| Injectability | Passes | N/D | N/D | N/D | N/D | N/D | 27G to |
| and | | | | | | | 30G |
| Syringeability | | | | | | | |
| Density | 1.01 | N/D | N/D | N/D | N/D | N/D | N/A |
| (g/mL) at RT | | | | | | | |
| Viscosity | 1.05 | N/D | N/D | 1.01 | 1.07 | 1.09 | N/A |
| (cP) | | | | | | | |
| Particulate | 226 | Passes | Passes | 88 | 108 | N/D | 10 μm ≤ |
| Matter | | visual | visual | | | | 6000 |
| | 10 | Passes | Passes | 10 | 14 | N/D | 25 μm ≤ |
| | | visual | visual | | | | 600 |

ND: not done

TABLE 50
Appearance of FS-5 and FS-6 Formulations at Three Months

| Sample ID | Appearance |
|---|---|
| FS-5_25° C._3M | Clear and transparent solution (no change from T0) |
| FS-5_40° C._3M | Clear and transparent solution (no change from T0) |
| FS-6_25° C._3M | Clear and transparent solution (no change from T0) |
| FS-6_40° C._3M | Clear and transparent solution (no change from T0) |

### Stability of Nanoemulsion Formulation

The FE-6 formulation was tested for stability for 3 months at 25° C. and 40° C. Formulation FE-6 was filled into type 1 clear glass vials that were stopped and crimp-sealed. The vials were then placed into 25° C. and 40° C. storage conditions (protected from light) for stability testing. Each sample was pulled and tested according to Table 51 below. The results of the stability experiments are summarized in Tables 52-54.

TABLE 51
Stability Time Points
Bumetanide SubQ formulation Stability Time Points/Condition:

| Storage Condition | T0 | 0.5M | 1M | 2M | 3M |
|---|---|---|---|---|---|
| 25° C. | A | C | C | C | C |
| 40° C. | | C | C | C | C |

Panel A (TO only): Was studied for appearance, pH, osmolality, injectability (27G-30G) as per method in the appendix, density/viscosity, and HPLC assay/impurities.
Panel C (0.5M, 1M, 2M, 3M timepoints): Was studied for appearance, pH, viscosity, HPLC assay/impurities, particulate matter by USP <788> method, and particle size distribution.

TABLE 52
Summary of FE-6 formulation at 25° C.
FE-6 @ 25° C. Stability Results

| Time Point | T0 | 0.5 M | 1 M | 2 M | 3 M | Comments |
|---|---|---|---|---|---|---|
| Appearance | White, milky | White, Milky | White, Milky | White, Milky | White, Milky | N/A |
| | emulsion | Emulsion | Emulsion | Emulsion | Emulsion | |
| Assay | 9.91 mg/mL | 9.91 mg/mL | 9.82 mg/mL | 9.78 mg/mL | 9.78 mg/mL | N/A |
| | 100% | 100.07% | 99.12% | 98.71% | 98.71% | N/A |
| Imp. A (RRT = 0.59 | <0.05% | <0.05% | <0.05% | <0.05% | <0.05% | N/A |
| mins) | | | | | | |
| Imp. B (RRT = 0.42 | <0.05% | 0.08% | 0.11% | 0.17% | 0.20% | N/A |
| mins) | | | | | | |
| Imp. C (RRT = 3.12 | <0.05% | N/D | N/D | N/D | N/D | N/A |
| mins) | | | | | | |
| Imp. D (RRT = 2.50 | <0.05% | N/D | N/D | N/D | N/D | N/A |
| mins) | | | | | | |
| Total Impurities (%) | 0.07% | 0.09% | 0.13% | 0.17% | 0.21% | N/A |
| pH | 7.0 | 7.1 | 6.9 | 7.0 | 7.0 | N/A |
| Osmolality (mOsm/kg) | 309 | N/D | N/D | N/D | N/D | N/A |
| Injectability and | Pass | N/D | N/D | N/D | N/D | 27G to |
| Syringeability | | | | | | 30G |
| Density (g/mL) at RT | 1.00 | N/D | N/D | N/D | N/D | N/A |
| Viscosity (cP) | 1.56 | 1.48 | 1.48 | 1.47 | 1.46 | N/A |
| Particulate Matter | N/D | 462 | 22 | 18 | 24 | 10 μm ≤ |
| | | | | | | 6000 |
| | N/D | 52 | 34 | 12 | 11 | 25 μm ≤ |
| | | | | | | 600 |
| Particle Size | Zavg: 100.3 nm | 109.2 | 111.7 | 117.2 | 118.6 | N/A |
| Distribution | PDI: 0.14 | 0.12 | 0.11 | 0.08 | 0.08 | N/A |
| | D(i; 0.1): 65.0 nm | 71.3 | 74.6 | 80.2 | 81.0 | N/A |
| | D(i; 0.5): 109.3 nm | 116.0 | 140.0 | 123.0 | 125.0 | N/A |
| | D(i; 0.9): 183.3 nm | 192.7 | 191.3 | 190.0 | 193.0 | N/A |

N/D: Not done

TABLE 53
Summary of FE-6 formulation at 40° C.
FE-6 @ 40° C. Stability Results

| Time Point | T0 | 0.5 M | 1 M | 2 M | 3 M | Comments |
|---|---|---|---|---|---|---|
| Appearance | White, milky | White, milky | White, milky | White, Milky | White, Milky | N/A |
| | emulsion | emulsion | emulsion | Emulsion | Emulsion | |
| Assay | 9.91 mg/mL | 9.87 mg/mL | 9.66 mg/mL | 9.76 mg/mL | 9.68 mg/mL | Assay in |
| | | | | | | mg/mL |
| | 100% | 99.61% | 97.58% | 98.55% | 97.72% | Recovery |
| | | | | | | % |
| Imp. A (RRT = 0.59 mins) | <0.05% | <0.05% | <0.05% | <0.05% | <0.05% | N/A |
| Imp. B (RRT = 0.42 mins) | <0.05% | 0.18% | 0.24% | 0.40% | 0.54% | N/A |
| Imp. C (RRT = 3.12 mins) | <0.05% | N/D | N/D | N/D | N/D | N/A |
| Imp. D (RRT = 2.50 mins) | <0.05% | N/D | N/D | N/D | N/D | N/A |
| Total Impurities (%) | 0.07% | 0.18% | 0.25% | 0.46% | 0.54% | N/A |
| pH | 7.0 | 7.0 | 6.8 | 6.9 | 6.7 | N/A |
| Osmolality (mOsm/kg) | 309 | N/D | N/D | N/D | N/D | N/A |
| Injectability and | Pass | N/D | N/D | N/D | N/D | 27G to |
| Syringeability | | | | | | 30G |
| Density (g/mL) at RT | 1.00 | N/D | N/D | N/D | N/D | N/A |
| Viscosity (cP) | 1.56 | 1.48 | 1.45 | 1.47 | 1.48 | N/A |
| Particulate Matter | N/D | 132 | 8 | 13 | 10 | 10 μm ≤ |
| | | | | | | 6000 |
| | N/D | 20 | 61 | 3 | 6 | 25 μm ≤ |
| | | | | | | 600 |
| Particle Size | Zavg: 100.3 nm | 113.1 | 113.6 | 117.9 | 121.9 | N/A |
| Distribution | PDI: 0.14 | 0.10 | 0.09 | 0.09 | 0.09 | N/A |
| | D(i; 0.1): 65.0 nm | 75.8 | 77.8 | 82.2 | 82.8 | N/A |
| | D(i; 0.5): 109.3 nm | 119.7 | 119.3 | 124.0 | 128.0 | N/A |
| | D(i; 0.9): 183.3 nm | 190.7 | 187.0 | 188.0 | 200.0 | N/A |

TABLE 54
Appearance of FE-6 Formulation at Three Months

| Sample ID | Appearance |
|---|---|
| FE-6_25° C._3M | White opaque emulsion (no change from T0) |
| FE-6_40° C._3M | White opaque emulsion (no change from T0) |

### Stability of CLEARSOL™ Formulation

The FC-2 formulation was made according to the compositions described in Table 55 below. The FC-2 formulation was performed by adding 0.8 g of ClearSol™ F95V into a 2 mL centrifuge vial. The pH was checked and adjusted to about 8.0 with 1 N NaOH. To this composition, 10 mg of bumetanide was added and mixed for about 30 seconds. The appearance was checked as well as the pH and was brought to a mass of 1 g with ClearSol F95V. 0.75 mL of the composition was added into a spin-x having a 0.22 μm filter and centrifuged at 13,000 rpm for 2 minutes. The filtrate was then transferred and diluted for characterization by High Performance Liquid Chromatography. The formulation was a clear pale, yellow liquid.

TABLE 55
ClearSol ™ Formulation Table

| Ingredient | FC-2 (%, w/w) |
|---|---|
| Bumetanide | 1.00 |
| Soy Lecithin (PL90G) | 20.00 |
| Glycocholic Acid (GCA) | 13.04 |
| MCT 812 | 6.40 |
| PEG300 | 9.85 |
| Sterile Water for Injection | 7.88 |
| Propylene glycol | Q.S. to 100 |
| Adjust pH to target with 1.0 NaOH or 1.0 | 7.0 |
| HCl | |

*Target pH (7.0 ± 0.5), adjusted pH with NaOH/HCl as needed

The FC-2 formulation was tested for stability for 3 months at 25° C. and 40° C. The formulation FC-2 was filled into type 1 clear glass vials that were stopped and crimp-sealed. The vials were then placed into 25° C. and 40° C. storage conditions (protected from light) for stability testing. Each sample was pulled and tested according to Table 56 below. The results of the stability experiments are summarized in Tables 57-59.

TABLE 56
Stability Time Points
Bumetanide SubQ formulation Stability Time Points/Condition:

| Storage Condition | T0 | 0.5M | 1M | 2M | 3M |
|---|---|---|---|---|---|
| 25° C. | A | B | B | B | B |
| 40° C. | | B | B | B | B |

Panel A (TO only): Was studied for appearance, pH, osmolality, injectability (27G-30G) as per method in the appendix, density/viscosity, HPLC assay/impurities, and particulate matter by USP <788> method.
Panel B (0.5M, 1M, 2M, 3M timepoints): Was studied for appearance, pH, viscosity, HPLC assay/impurities, and particulate matter by USP <788> method.

TABLE 57
Summary of FC-2 formulation at 25° C.
FC-2 @ 25° C. Stability Results

| Time Point | T0 | 0.5 M | 1 M | 2 M | 3 M | Comments |
|---|---|---|---|---|---|---|
| Appearance | Slightly yellow | Slightly yellow | Slightly yellow | Slightly yellow | Slightly yellow | N/A |
| | transparent | transparent | transparent | transparent | transparent | |
| | solution | solution | solution | solution | solution | |
| Assay | 10.02 mg/ml | 9.93 mg/mL | 9.82 mg/mL | 9.99 mg/mL | 10.07 mg/mL | N/A |
| | 100% | 99.17% | 98.04% | 99.80% | 100.50% | N/A |
| Imp. A (RRT = | <0.05% | <0.05% | <0.05% | <0.05% | <0.05% | N/A |
| 0.59 mins) | | | | | | |
| Imp. B (RRT = | <0.05% | <0.05% | <0.05% | 0.08% | 0.10% | N/A |
| 0.42 mins) | | | | | | |
| Imp. C (RRT = | N/D | N/D | N/D | N/D | N/D | N/A |
| 3.12 mins) | | | | | | |
| Imp. D (RRT = | N/D | N/D | N/D | N/D | N/D | N/A |
| 2.50 mins) | | | | | | |
| Unknown | <0.05% | <0.05% | <0.05% | <0.05% | <0.05% | N/A |
| Imp. I (RRT = | | | | | | |
| 0.41 mins) | | | | | | |
| Unknown | N/D | <0.05% | <0.05% | <0.05% | <0.05% | N/A |
| Imp. I (RRT = | | | | | | |
| 0.69 mins) | | | | | | |
| Total | 0.05% | 0.03% | 0.05% | 0.14% | 0.15% | N/A |
| Impurities | | | | | | |
| (%) | | | | | | |
| pH | 6.9 | 6.6 | 6.9 | 6.8 | 6.7 | N/A |
| Osmolality | Refer to | N/D | N/D | N/D | N/D | N/A |
| (mOsm/kg) | FootNote | | | | | |
| Injectability | Pass | N/D | N/D | N/D | N/D | 27G to |
| and | | | | | | 30G |
| Syringeability | | | | | | |
| Density | 1.03 | N/D | N/D | N/D | N/D | N/A |
| (g/mL) at RT | | | | | | |
| Viscosity | 205.40 | 204.0 | 205.7 | 209.9 | 206.0 | N/A |
| (cP) @ | | | | | | |
| 10 RPM | | | | | | |
| Particulate | 1352 | Visual | Visual | Visual | Visual | 10 μm ≤ |
| Matter | | Passes | Passes | Passes | Passes | 6000 |
| | 40 | Visual | Visual | Visual | Visual | 25 μm ≤ |
| | | Passes | Passes | Passes | Passes | 600 |

N/D: not detected or not done

TABLE 58
Summary of FC-2 formulation at 40° C.
FC-2 @ 40° C. Stability Results

| Time Point | T0 | 0.5 M | 1 M | 2 M | 3 M | Comments |
|---|---|---|---|---|---|---|
| Appearance | Slightly yellow | Slightly yellow | Yellow | Yellow | Yellow | N/A |
| | transparent | transparent | transparent | transparent | transparent | |
| | solution | solution | solution | solution | solution | |
| Assay | 10.02 mg/ml | 9.88 mg/mL | 9.76 mg/mL | 9.76 mg/mL | 9.53 mg/mL | N/A |
| | 100% | 98.60% | 97.40% | 97.41% | 98.37% | N/A |
| Imp. A (RRT = | <0.05% | <0.05% | 0.05% | 0.07% | <0.05% | N/A |
| 0.59 mins) | | | | | | |
| Imp. B (RRT = | <0.05% | 0.11% | 0.22% | 0.27% | 0.33% | N/A |
| 0.42 mins) | | | | | | |
| Imp. C (RRT = | <0.05% | N/D | N/D | N/D | N/D | N/A |
| 3.12 mins) | | | | | | |
| Imp. D (RRT = | <0.05% | N/D | N/D | N/D | N/D | N/A |
| 2.50 mins) | | | | | | |
| Unknown Imp. | <0.05% | 0.05% | 0.10% | 0.11% | 0.17% | N/A |
| I (RRT = | | | | | | |
| 0.41 mins) | | | | | | |
| Unknown Imp. | N/D | <0.05% | 0.09% | 0.18% | 0.14% | N/A |
| I (RRT = | | | | | | |
| 0.69 mins) | | | | | | |
| Total | 0.05% | 0.23% | 0.46% | 0.63% | 0.67% | N/A |
| Impurities (%) | | | | | | |
| pH | 6.9 | 6.6 | 6.8 | 6.7 | 6.7 | N/A |
| Osmolality | Refer to | N/D | N/D | N/D | N/D | N/A |
| (mOsm/kg) | FootNote | | | | | |
| Injectability | Pass | N/D | N/D | N/D | N/D | 27G to |
| and | | | | | | 30G |
| Syringeability | | | | | | |
| Density | 1.03 | N/D | N/D | N/D | N/D | N/A |
| (g/mL) at RT | | | | | | |
| Viscosity (cP) | 205.40 | 207.0 | 205.4 | 205.7 | 203.7 | N/A |
| @ 10 RPM | | | | | | |
| Particulate | 1352 | Visual | Visual | Visual | Visual | 10 μm ≤ |
| Matter | | Passes | Passes | Passes | Passes | 6000 |
| | 40 | Visual | Visual | Visual | Visual | 25 μm ≤ |
| | | Passes | Passes | Passes | Passes | 600 |

N/D: not detected or not done

TABLE 59
Appearance of FE-6 Formulation at Three Months

| Sample ID | Appearance |
|---|---|
| FC-2_T0 | Slightly yellow transparent solution |
| FC-2_25° C._3M | Slightly yellow transparent solution |
| | (no change from T0) |
| FC-2_40° C._3M | Yellow transparent solution |

The arginine salt formulations FS-5 and FS-6 showed good stability over 3 months at both room temperature and under accelerated condition (40° C.). The potassium salt formulations FS-3 and FS-4 showed poor appearance with needle like precipitation after 1 month at room temperature condition. The nano-emulsion formulation, FE-6, showed a significant increase in degradation impurities over 3 months at both room temperature and under accelerated condition (40° C.). The Clearsol formulation, FC-2, showed a significant increase in color formation and degradation impurities after 3 months under accelerated conditions (40° C.)

### Example 15. Administration of Salts of Bumetanide

The purpose of this study was to assess the pharmacokinetic profiles of various formulations of bumetanide, including arginine bumetanide salts (FS-5 and FS-6) and potassium bumetanide salts (FS-3 and FS-4) for subcutaneous administration, and an bumetanide intranasal formulation (F82), which are described in Table 60, when each formulation is administered as a single subcutaneous, or intranasal in different vehicles when compared to single IV administration dose in male and female beagle dogs.

A total of four dogs were assigned to the study (3 males, 9-13 kg and 1 female, 7-10 kg) ranging from 1 to 3 years old.

The study was conducted in 6 Events in the same animals, separated by at least 48 hours or more washout period between events.

For subcutaneous administration the bumetanide formulations were injected subcutaneously (100 μL/dog), using a 25G hypodermic needle. After each application, successful dosing was verified by visual inspection of the dog injection site and after 1 minute of dosing. Only successful administrations will be accepted.

For intranasal administration, each dog was held in an upright position with the nose pointing up. The test article (50 μL) was delivered into each nostril (total 100 μL/dog). The nose was held pointing up for ˜60 seconds after dosing. After each application, successful dosing was verified by visual inspection of the pipette tip and the dog nostril.

For intravenous administration, the commercially available injection of bumetanide (Walgreens, 0.25 mg/mL) was used and administered via the cephalic vein over a period of 30 seconds (4.0 mL/dog, IV).

Body weights of the dogs were measured for each dog prior to each dose on the day of dose, and approximately 48 hours post-dose for each event. For some events, the 48 hour post-dose weight was the same weight as the pre-dose weight for the following dose. Urine production was also monitored post-dose to assess diuresis by making observations at 30 minutes, 1, 2, 3 and 4 hours post-dose. Urine volume was not measured. On each day of dosing, blood (˜600 μL/timepoint, jugular or cephalic vein) were collected in red top tubes with clotting activator (Sarstedt, Inc-41.1392.105 or similar) to obtain serum at the times described in Table 61. Blood was centrifuged at 5000×g for 10 minutes at 4° C. If the samples were collected outside the collection window, they were not be considered a protocol deviation. Serum was divided into 2 aliquots of ˜150 μL each and stored frozen at −20° C. until it was shipped to Climax Laboratories, Inc. on dry ice.

TABLE 60
Bumetanide formulations administered to dogs

| | | | | | | | Dose |
|---|---|---|---|---|---|---|---|
| | | | | | | Dose | Concentration |
| | | | | | Dose | Volume | (mg/mL) |
| | Animal | Test | | Dose | Level | or Dose | or dose |
| Event | # | Article | Formulation | Route | (mg/dog) | weight | strength |
| 1 | 1501 | Bumetanide | IV Injection | IV | 1.0 | 4.0 | 0.25 |
| | 1001 | | | | | mL/animal | mg/mL |
| | 1002 | | | | | | |
| | 1003 | | | | | | |
| 2 | 1501 | RSQ101 | Formulation FS-4 | SQ | 1.01 | 100 | 10.1 |
| | 1001 | | | | | μL/animal | mg/mL |
| | 1002 | | | | | | |
| | 1003 | | | | | | |
| 3 | 1501 | RSQ101 | Formulation FS-3 | SQ | 1.01 | 100 | 10.1 |
| | 1001 | | | | | μL/animal | mg/mL |
| | 1002 | | | | | | |
| | 1003 | | | | | | |
| 4 | 1501 | RSQ101 | Formulation FS-5 | SQ | 0.97 | 100 | 9.7 |
| | 1001 | | | | | μL/animal | mg/mL |
| | 1002 | | | | | | |
| | 1003 | | | | | | |
| 5 | 1501 | RSQ101 | Formulation FS-6 | SQ | 1.0 | 100 | 10 |
| | 1001 | | | | | μL/animal | mg/mL |
| | 1002 | | | | | | |
| | 1003 | | | | | | |
| 6 | 1501 | RSQ101 | Formulation F82 | Intra-nasal | 1.0 | 100 | 10 |
| | 1001 | | (pH 6.6) | | | μL/animal | mg/mL |
| | 1002 | | | | | | |
| | 1003 | | | | | | |

TABLE 61
Collection times and windows of serum

| Event 1 | | | | | |
|---|---|---|---|---|---|
| Collection | | Events 2-5 | | Event 6 | |
| Time | Collection | Collection | Collection | Collection | Collection |
| [symbol] | [symbol] | [symbol] | [symbol] | [symbol] | [symbol] |
| 2 | ±1 min | Pre-dose | Prior to | Pre-dose | Prior to |
| | | | dosing | | dosing |
| 5 | ±1 min | 2 | ±1 min | 5 | ±1 min |
| 10 | ±2 min | 5 | ±1 min | 10 | ±2 min |
| 15 | ±2 min | 10 | ±2 min | 15 | ±2 min |
| 20 | ±3 min | 15 | ±2 min | 20 | ±3 min |
| 30 | ±3 min | 20 | ±3 min | 30 | ±3 min |
| 45 | ±3 min | 30 | ±3 min | 45 | ±3 min |
| 60 | ±4 min | 45 | ±3 min | 60 | ±4 min |
| 120 | ±5 min | 60 | ±4 min | 120 | ±5 min |
| 180 | ±5 min | 120 | ±5 min | 180 | ±5 min |
| — | — | 180 | ±5 min | 240 | ±5 min |
| — | — | 240 | ±5 min | — | — |

[symbol]

indicates data missing or illegible when filed

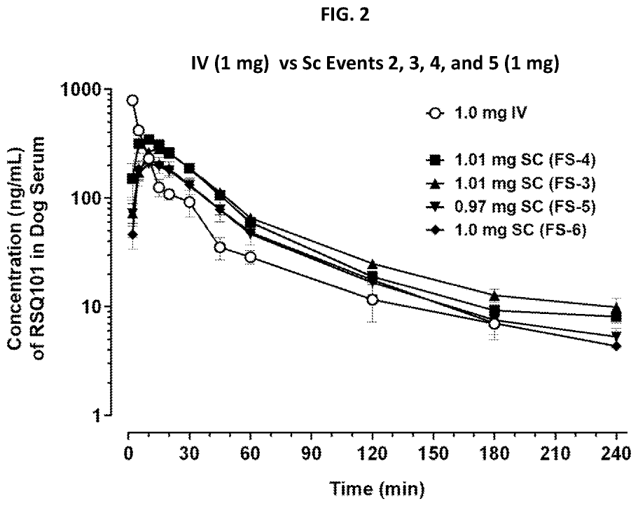

Individual serum concentrations and pharmacokinetic parameters of bumetanide following IV administration of commercially available bumetanide to dogs are shown in Table 62 and presented graphically in FIGS. 2-4. Following IV administration of bumetanide, levels of bumetanide also declined rapidly.

TABLE 62
Pharmacokinetic parameters of bumetanide following IV administration

| | Dose (mg) | | | | | |
|---|---|---|---|---|---|---|
| | 1 | 1 | 1 | 1 | | |
| | Route | | | | | |
| | IV | IV | IV | IV | | |
| | Event | | | | | |
| | 1 | 1 | 1 | 1 | | |
| | Animal ID | | | | | |
| Time (min) | 1501 | 1001 | 1002 | 1003 | mean | SD |
| 0 | 0 | 0 | 0 | 0 | | |
| 2 | 723 | 831 | 841 | 768 | 791 | 55.5 |
| 5 | 388 | 429 | 454 | 403 | 419 | 29.1 |
| 10 | 197 | 245 | 246 | 233 | 230 | 22.9 |
| 15 | 112 | 68.8 | 150 | 171 | 125 | 45.0 |
| 20 | 74.7 | 114 | 111 | 135 | 109 | 25.0 |
| 30 | 42.0 | 159 | 65.3 | 102 | 92.1 | 51.0 |
| 45 | 21.5 | 34.9 | 26.3 | 58.4 | 35.3 | 16.4 |
| 60 | 16.7 | 31.6 | 31.6 | 34.8 | 28.7 | 8.12 |
| 120 | 5.26 | 24.5 | 6.71 | 10.0 | 11.6 | 8.82 |
| 180 | 2.29 | 12.1 | 6.62 | 6.91 | 6.98 | 4.01 |
| AUC (ng*min/mL) | 8418 | 13038 | 10996 | 12005 | 11114 | 1982 |
| AUC (ng*h/mL) | 140 | 217 | 183 | 200 | 185 | 33.0 |
| Weight (kg) | 9.1 | 9.9 | 9.8 | 9.6 | 9.60 | 0.36 |
| Dose (mg) | 1.000 | 1.000 | 1.000 | 1.000 | 1.000 | 0.000 |
| Dose (mg/kg) | 0.1099 | 0.1010 | 0.1020 | 0.1042 | 0.1043 | 0.0040 |
| DA AUC | 1277 | 2151 | 1796 | 1921 | 1786 | 370 |
| (ng*h/mL)/(mg/kg) | | | | | | |
| DA AUC | 128 | 215 | 180 | 192 | 179 | 37.0 |
| (ng*h/mL)/10 kg | | | | | | |
| dog | | | | | | |

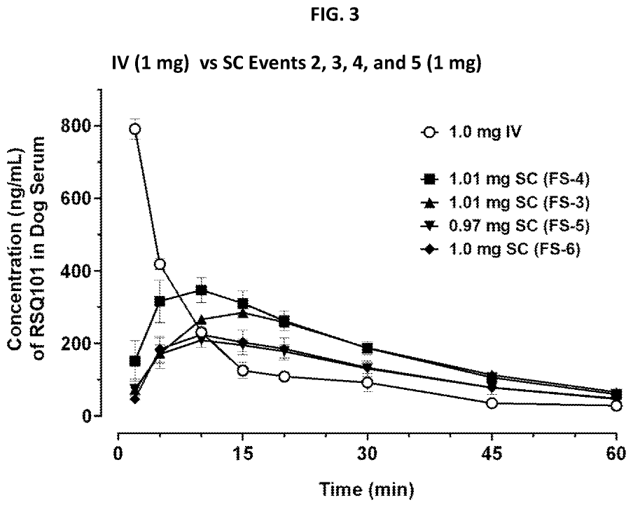

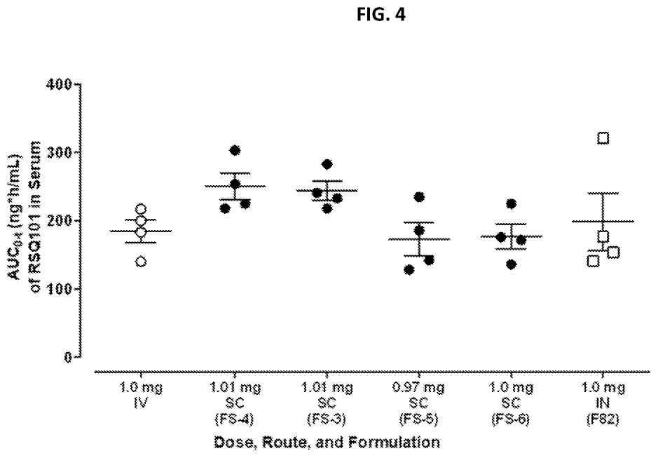

Individual serum concentrations and pharmacokinetic parameters of bumetanide following subcutaneous administration of potassium bumetanide formulations are shown in Tables 63 and 64. When the bumetanide formulation was delivered subcutaneously, the potassium bumetanide formulations, FS-4 and FS-3, resulted in the greatest absorption of bumetanide as shown in FIGS. 2-4. Subcutaneous administration of potassium bumetanide resulted in higher serum concentrations compared to IV administration of bumetanide as is shown in FIGS. 2 and 3. Subcutaneous administration resulted in rapid administration of FS-4 and FS-3 and more persistent levels over time in comparison to IV administration as is shown FIG. 3. Formulations FS-3 and FS-4 showed remarkably higher AUC (FIG. 4) compared to FS-5 and FS-6 arginine salt formulations and IV administration.

TABLE 63
Pharmacokinetic parameters of FS-3 following subcutaneous administration

| | Dose (mg) | | | | | |
|---|---|---|---|---|---|---|
| | 1.01 | 1.01 | 1.01 | 1.01 | | |
| | Route | | | | | |
| | SQ FS-3 | SQ FS-3 | SQ FS-3 | SQ FS-3 | | |
| | Event | | | | | |
| | 2 | 2 | 2 | 2 | | |
| | Animal ID | | | | | |
| Time (min) | 1501 | 1001 | 1002 | 1003 | mean | SD |
| 0 | 0 | 0 | 0 | 0 | | |
| 2 | 46.3 | 31.2 | 54.2 | 160 | 72.9 | 58.8 |
| 5 | 232 | 211 | 200 | 45.9 | 172 | 85.3 |
| 10 | 295 | 252 | 264 | 252 | 266 | 20.3 |
| 15 | 315 | 274 | 259 | 289 | 284 | 23.9 |
| 20 | 295 | 250 | 232 | 255 | 258 | 26.6 |
| 30 | 207 | 200 | 158 | 185 | 188 | 21.7 |
| 45 | 119 | 118 | 90.6 | 125 | 113 | 15.3 |
| 60 | 75.5 | 57.9 | 60.7 | 68.6 | 65.7 | 7.96 |
| 120 | 32.5 | 23.5 | 20.4 | 23.5 | 25.0 | 5.23 |
| 180 | 17.0 | 8.20 | 12.10 | 13.6 | 12.7 | 3.65 |
| 240 | 16.0 | 6.51 | 8.01 | 9.02 | 9.89 | 4.21 |
| Cmax (ng/mL) | 315 | 274 | 264 | 289 | 286 | 22.2 |
| Tmax (min) | 15 | 15 | 10 | 15 | 15 | |
| AUC (ng*min/mL) | 16960 | 13966 | 13091 | 14458 | 14619 | 1660 |
| AUC (ng*h/mL) | 283 | 233 | 218 | 241 | 244 | 27.7 |
| Weight (kg) | 8.9 | 9.8 | 9.6 | 9.4 | 9.43 | 0.386 |
| Dose (mg) | 1.010 | 1.010 | 1.010 | 1.010 | 1.010 | 0.000 |
| Dose (mg/kg) | 0.1135 | 0.1031 | 0.1052 | 0.1074 | 0.1073 | 0.0045 |
| DA AUC | 2491 | 2258 | 2074 | 2243 | 2266 | 171 |
| (ng*h/mL)/(mg/kg) | | | | | | |
| DA AUC | 264 | 214 | 218 | 244 | 235 | 23.6 |
| (ng*h/mL)/10 kg | | | | | | |
| dog | | | | | | |
| Bioavailability | 208% | 109% | 123% | 124% | 141% | 45.2% |

TABLE 64
Pharmacokinetic parameters of FS-4 following subcutaneous administration

| | Dose (mg) | | | | | |
|---|---|---|---|---|---|---|
| | 1.01 | 1.01 | 1.01 | 1.01 | | |
| | Route | | | | | |
| | SQ FS-4 | SQ FS-4 | SQ FS-4 | SQ FS-4 | | |
| | Event | | | | | |
| | 2 | 2 | 2 | 2 | | |
| | Animal ID | | | | | |
| Time (min) | 1501 | 1001 | 1002 | 1003 | mean | SD |
| 0 | 0 | 0 | 0 | 0 | | |
| 2 | 261 | 65.9 | 233 | 45.3 | 151 | 111 |
| 5 | 398 | 190 | 433 | 244 | 316 | 118 |
| 10 | 404 | 260 | 404 | 319 | 328 | 72.4 |
| 15 | 410 | 263 | 279 | 288 | 310 | 67.5 |
| 20 | 339 | 247 | 210 | 251 | 262 | 54.7 |
| 30 | 224 | 180 | 139 | 205 | 187 | 36.7 |
| 45 | 132 | 108 | 76.4 | 108 | 106 | 22.8 |
| 60 | 73.4 | 55.3 | 44.4 | 66.0 | 59.8 | 12.7 |
| 120 | 16.3 | 17.5 | 16.4 | 25.8 | 19.0 | 4.57 |
| 180 | 10.7 | 7.4 | 7.15 | 11.7 | 9.23 | 2.31 |
| 240 | 6.30 | 8.19 | 7.08 | 10.8 | 8.09 | 1.96 |
| Cmax (ng/mL) | 410 | 263 | 433 | 319 | 356 | 79.3 |
| Tmax (min) | 15 | 15 | 5 | 10 | 11.3 | 4.8 |
| AUC (ng*min/mL) | 18199 | 13074 | 13478 | 15238 | 14997 | 2332 |
| AUC (ng*h/mL) | 303 | 218 | 225 | 254 | 250 | 38.9 |
| Weight (kg) | 8.7 | 9.8 | 9.7 | 9.6 | 9.5 | 0.5 |
| Dose (mg) | 1.010 | 1.010 | 1.010 | 1.010 | 1.010 | 0.000 |
| Dose (mg/kg) | 0.1161 | 0.1031 | 0.1041 | 0.1052 | 0.1071 | 0.0060 |
| DA AUC | 2613 | 2114 | 2157 | 2414 | 2325 | 233 |
| (ng*h/mL)/(mg/kg) | | | | | | |
| DA AUC | 264 | 214 | 218 | 244 | 235 | 23.6 |
| (ng*h/mL)/10 kg | | | | | | |
| dog | | | | | | |
| Bioavailability | 228% | 102% | 125% | 128% | 146% | 56.1% |

Individual serum concentrations and pharmacokinetic parameters of bumetanide following subcutaneous administration of arginine bumetanide formulations are shown in Tables 65 and 66. When the bumetanide formulation was delivered subcutaneously, the arginine bumetanide formulations, FS-5 and FS-6, resulted in the absorption of bumetanide less than the potassium bumetanide formulations as shown in FIGS. 2-4.

TABLE 65
Pharmacokinetic parameters of FS-5 following subcutaneous administration

| | Dose (mg) | | | | | |
|---|---|---|---|---|---|---|
| | 0.97 | 0.97 | 0.97 | 0.97 | | |
| | Route | | | | | |
| | SQ FS-5 | SQ FS-5 | SQ FS-5 | SQ FS-5 | | |
| | Event | | | | | |
| | 4 | 4 | 4 | 4 | | |
| | Animal ID | | | | | |
| Time (min) | 1501 | 1001 | 1002 | 1003 | mean | SD |
| 0 | 0 | 0 | 0 | 0 | | |
| 2 | 90.5 | 110 | 44.3 | 43.0 | 72.0 | 33.6 |
| 5 | 158 | 247 | 136 | 141 | 171 | 51.9 |
| 10 | 182 | 262 | 176 | 215 | 209 | 39.4 |
| 15 | 180 | 224 | 154 | 22 | 195 | 33.8 |
| 20 | 159 | 182 | 143 | 228 | 178 | 37.0 |
| 30 | 96.6 | 133 | 101 | 191 | 130 | 43.5 |
| 45 | 51.7 | 69.3 | 61.0 | 130 | 78.0 | 35.4 |
| 60 | 31.3 | 40.7 | 39.4 | 75.7 | 46.8 | 19.7 |
| 120 | 6.00 | 21.7 | 16.3 | 22.8 | 16.7 | 7.68 |
| 180 | 3.61 | 7.49 | 6.02 | 13.0 | 7.53 | 3.98 |
| 240 | 2.99 | 4.68 | 5.36 | 8.10 | 5.28 | 2.13 |
| Cmax (ng/mL) | 182 | 262 | 176 | 228 | 212 | 40.6 |
| Tmax (min) | 10 | 10 | 10 | 20 | 10 | |
| AUC (ng*min/mL) | 7684 | 11178 | 8532 | 14129 | 10381 | 2908 |
| AUC (ng*h/mL) | 128 | 186 | 142 | 235 | 173 | 48.5 |
| Weight (kg) | 8.7 | 9.9 | 10.0 | 9.7 | 9.58 | 0.597 |
| Dose (mg) | 0.970 | 0.970 | 0.970 | 0.970 | 0.970 | 0.000 |
| Dose (mg/kg) | 0.1115 | 0.0980 | 0.0970 | 0.1000 | 0.1016 | 0.0067 |
| DA AUC | 1149 | 1901 | 1466 | 2355 | 1718 | 525 |
| (ng*h/mL)/(mg/kg) | | | | | | |
| DA AUC | 111 | 184 | 142 | 228 | 167 | 50.9 |
| (ng*h/mL)/10 kg | | | | | | |
| dog | | | | | | |
| Bioavailability | 93% | 83% | 74% | 113% | 90.6% | 16.8% |

TABLE 66
Pharmacokinetic parameters of FS-6 following subcutaneous administration

| | Dose (mg) | | | | | |
|---|---|---|---|---|---|---|
| | 1 | 1 | 1 | 1 | 1 | |
| | Route | | | | | |
| | SQ FS-6 | SQ FS-6 | SQ FS-6 | SQ FS-6 | SQ FS-6 | |
| | Event | | | | | |
| | 5 | 5 | 5 | 5 | 5 | |
| | Animal ID | | | | | |
| Time (min) | 1501 | 1001 | 1002 | 1003 | mean | SD |
| 0 | 0 | 0 | 0 | 0 | | |
| 2 | 66.9 | 36.4 | 66.9 | 15.6 | 46.5 | 25.1 |
| 5 | 236 | 155 | 249 | 96.5 | 184 | 71.7 |
| 10 | 288 | 198 | 270 | 138 | 224 | 69.0 |
| 15 | 286 | 185 | 217 | 123 | 203 | 67.8 |
| 20 | 263 | 176 | 186 | 114 | 185 | 61.1 |
| 30 | 169 | 141 | 124 | 97.1 | 133 | 30.2 |
| 45 | 92.7 | 92.6 | 66.1 | 62.7 | 78.5 | 16.4 |
| 60 | 61.9 | 49.9 | 39.7 | 42.3 | 48.5 | 10.0 |
| 120 | 19.1 | 17.4 | 14.1 | 20.0 | 17.7 | 2.60 |
| 180 | 7.45 | 7.25 | 5.19 | 8.30 | 7.05 | 1.32 |
| 240 | 4.86 | 4.18 | 3.87 | 4.44 | 4.34 | 0.419 |
| Cmax (ng/mL) | 288 | 198 | 270 | 138 | 224 | 69 |
| Tmax (min) | 10 | 10 | 10 | 10 | 10 | |
| AUC (ng*min/mL) | 13517 | 10573 | 10297 | 8157 | 10636 | 2203 |
| AUC (ng*h/mL) | 225 | 176 | 172 | 136 | 177 | 36.7 |
| Weight (kg) | 8.6 | 9.9 | 9.6 | 9.5 | 9.40 | 0.560 |
| Dose (mg) | 1.000 | 1.000 | 1.000 | 1.000 | 1.000 | 0.000 |
| Dose (mg/kg) | 0.1163 | 0.1010 | 0.1042 | 0.1053 | 0.1067 | 0.0066 |
| DA AUC | 1937 | 1745 | 1648 | 1291 | 1655 | 271 |
| (ng*h/mL)/(mg/kg) | | | | | | |
| DA AUC | 194 | 174 | 165 | 129 | 166 | 27.1 |
| (ng*h/mL)/10 kg | | | | | | |
| dog | | | | | | |
| Bioavailability | 170% | 81% | 96% | 69% | 104% | 45.4% |

Individual serum concentrations and pharmacokinetic parameters of bumetanide following intranasal administration of a bumetanide formulation is shown in Table 67. When the bumetanide formulation was delivered intranasally, the bumetanide formulation, F82, resulted in the absorption of bumetanide similar to the arginine bumetanide formulations as shown in FIG. 4.

TABLE 67
Pharmacokinetic parameters of F82 following intranasal administration

| | Dose (mg) | | | | | |
|---|---|---|---|---|---|---|
| | 1 | 1 | 1 | 1 | | |
| | Route | | | | | |
| | IN F82 | IN F82 | IN F82 | IN F82 | | |
| | Event | | | | | |
| | 6 | 6 | 6 | 6 | | |
| | Animal ID | | | | | |
| Time (min) | 1501 | 1001 | 1002 | 1003 | mean | SD |
| 0 | 0 | 0 | 0 | 0 | | |
| 5 | 383 | 405 | 442 | 356 | 397 | 36.4 |
| 10 | 238 | 288 | 115 | 436 | 269 | 133 |
| 15 | 94.9 | 89.5 | 65.7 | 231 | 120 | 74.9 |
| 20 | 81.2 | 115 | 49.8 | 121 | 91.8 | 33.0 |
| 30 | 71.0 | 111 | 33.1 | 138 | 88.3 | 45.9 |
| 45 | 61.9 | 56.5 | 33.0 | 122 | 68.4 | 37.9 |
| 60 | 43.4 | 41.6 | 50.1 | 97.3 | 58.1 | 26.4 |
| 120 | 15.1 | 21.1 | 19.0 | 64.2 | 29.9 | 23.0 |
| 180 | 8.11 | 10.3 | 12.3 | 27.7 | 14.6 | 8.90 |
| 240 | 7.25 | 6.30 | 9.37 | 17.6 | 10.1 | 5.14 |
| Cmax (ng/mL) | 383 | 405 | 442 | 436 | 417 | 28 |
| Tmax (min) | 5 | 5 | 5 | 10 | 5 | |
| AUC (ng*min/mL) | 9242 | 10643 | 8434 | 19268 | 11897 | 4998 |
| AUC (ng*h/mL) | 154 | 177 | 141 | 321 | 198 | 83.3 |
| Weight (kg) | 8.7 | 10.1 | 9.9 | 9.5 | 9.55 | 0.619 |
| Dose (mg) | 1.000 | 1.000 | 1.000 | 1.000 | 1.000 | 0.000 |
| Dose (mg/kg) | 0.1149 | 0.0990 | 0.1010 | 0.1053 | 0.1051 | 0.0071 |
| DA AUC | 1340 | 1792 | 1392 | 3051 | 1894 | 797 |
| (ng*h/mL)/(mg/kg) | | | | | | |
| DA AUC | 134 | 179 | 139 | 305 | 189 | 79.7 |
| (ng*h/mL)/10 kg | | | | | | |
| dog | | | | | | |
| Bioavailability | 115% | 80% | 76% | 162% | 108% | 40.0% |

The pharmacokinetic data collected upon administration of bumetanide formulations to dogs is summarized in Table 68. These data show the bioavailability and systemic exposure of bumetanide was high following subcutaneous and intranasal administration.

TABLE 68
Summary of pharmacokinetic parameters for bumetanide formulations

| | Route, Group, and | | AUC₀₋ₜ | |
|---|---|---|---|---|
| Event | Dose | Cₘₐₓ(ng/ml) | (ng*h/mL) | Bioavailability |
| 1 | 1.0 mg IV | NA | 185 | NA |
| 2 | 1.01 mg SC (FS-4) | 356 | 250 | 146% |
| 3 | 1.01 mg SC (FS-3) | 286 | 244 | 141% |
| 4 | 0.97 mg SC (FS-5) | 212 | 173 | 90.6% |
| 5 | 1.0 mg SC (FS-6) | 224 | 177 | 104% |
| 6 | 1.0 mg IN (F82) | 417 | 198 | 108% |

### Example 16. Administration of a Subcutaneous Potassium Salt of Bumetanide to a Patient Suffering Congestive Heart Failure with Severe Edema Caused Inadequate Gastrointestinal Absorption

According to the methods described herein, a physician of skill in the art can treat a patient, such as a human patient, so as to reduce or alleviate symptoms of edema arising from congestive heart failure. To this end, a physician of skill in the art has the patient administer to themselves a potassium salt of bumetanide or administers the potassium salt of bumetanide themselves. The potassium salt of bumetanide includes the formulation of FS-3 or FS-4. The potassium salt of bumetanide is administered by or to the patient experiencing symptoms of congestive heart failure such as shortness of breath, fatigue or edema that not reduced with the patient's typical daily dosage of an oral diuretic. At this time, the patient is administered the potassium salt of bumetanide subcutaneously in an amount of between 50 μL to 100 μL. Typical dosages are administered based on body weight, and are in the range of about 0.5-10 mg of the potassium salt of bumetanide over a 12 hour period, and not exceeding 10 mg of the potassium salt of bumetanide over a 12 hour period without consulting a physician of skill in the art. The subcutaneous formulation of the potassium salt is administered in one, two, three, four five, or 6 doses over a 4 hour period.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. This application claims the benefit of U.S. Provisional Application No. 63/208,863, filed Jun. 9, 2021, which is incorporated herein by reference in its entirety.

Other embodiments are in the claims.

What is claimed is:

1. A method of treating edema in a subject, the method comprising administering subcutaneously an effective amount of a pharmaceutical composition to the subject comprising (i) an aqueous solution having a pH of between about 5 and about 9, (ii) between about 4 mg/ml and about 20 mg/mL arginine bumetanide salt, and (iii) one or more pharmaceutically acceptable excipients.

2. The method of claim 1, wherein the pharmaceutical composition comprises (i) an aqueous solution having between about 5 mg/mL and about 12 mg/mL arginine bumetanide salt, (ii) one or more pharmaceutically acceptable excipients, and (iii) wherein the aqueous solution has a pH of between about 6 and about 8.

3. The method of claim 1 or 2, wherein the pharmaceutical composition comprises a buffering agent.

4. The method of claim 3, wherein the aqueous solution is free of a buffering agent other than the buffer formed by bumetanide free acid combined with arginine.

5. A method of treating edema in a subject, the method comprising administering subcutaneously an effective amount of a pharmaceutical composition to the subject comprising (i) an aqueous solution having a pH of between about 5 and about 9, (ii) between about 4 mg/mL and about 20 mg/mL potassium bumetanide salt, and (iii) one or more pharmaceutically acceptable excipients.

6. The method of claim 5, wherein the pharmaceutical composition comprises (i) an aqueous solution having between about 5 mg/mL and about 12 mg/mL potassium bumetanide salt, (ii) one or more pharmaceutically acceptable excipients, and (iii) wherein the aqueous solution has a pH of between about 6 and about 8.

7. The method of claim 5 or 6, wherein the pharmaceutical composition comprises a buffering agent.

8. The method of claim 7, wherein the aqueous solution is free of a buffering agent other than the buffer formed by bumetanide free acid combined with arginine potassium hydroxide.

9. The method of any one of claims 1-8, wherein the pharmaceutically acceptable excipients comprise a tonicity agent.

10. The method of any one of claims 1-9, wherein the aqueous solution has a pH of between about 6 and about 8.

11. The method of any one of claims 1-10, wherein the one or more pharmaceutically acceptable excipients comprises a sugar or sugar alcohol.

12. The method of claim 11, wherein the pharmaceutical composition comprises sucrose and/or mannitol.

13. The method of any one of claims 1-12, wherein the one or more pharmaceutically acceptable excipients comprises a preservative.

14. The method of claim 13, wherein the pharmaceutical composition comprises benzyl alcohol.

15. The method of any one of claims 1-14, wherein the pharmaceutical composition is administered in a dose volume of no more than 0.5 mL.

16. The method of claim 15, wherein the pharmaceutical composition is administered in a dose volume of between 25 μl to 250 μl.

17. The method of any one of claims 1-16, wherein pharmaceutical composition has a bioavailability of at least 90% upon administration to the subject.

18. The method claim 17, wherein the pharmaceutical composition has a bioavailability of at least 95% upon administration to the subject.

19. The method of any one of claims 1-18, wherein the edema is refractory to oral diuretics.

20. The method of any one of claims 1-19, wherein the subject has congestive heart failure.

21. The method of any one of claims 1-20, wherein the subject is suffering from edema in the lung.

22. The method of any one of claims 1-21, wherein the pharmaceutical composition is administered subcutaneously to the subject in an outpatient setting or the pharmaceutical composition is self-administered.

23. The method of any one of claims 1-22, wherein the subject has failed to achieve diuresis with oral diuretic therapy prior to the administering.

24. The method of any one of claims 1-23, wherein the subject is experiencing swelling of the legs, shortness of breath, difficulty breathing, or chest pain unresolved with oral diuretic therapy prior to the administering.

25. The method of any one of claims 1-24, wherein the subject is experiencing reduced intestinal motility prior to the administering.

26. A pharmaceutical composition comprising an aqueous solution arginine bumetanide formulated for subcutaneous administration having a pH of between about 6 and about 8, between about 5 mg/mL and 12 mg/mL, and pharmaceutical excipients comprising a sugar or a sugar alcohol.

27. The pharmaceutical composition of claim 25, wherein the arginine bumetanide has a concentration of between about 8 mg/ml and about 10 mg/mL.

28. A pharmaceutical composition comprising an aqueous solution potassium bumetanide formulated for subcutaneous administration having a pH of between about 6 and about 8, between about 5 mg/mL and 12 mg/mL, and pharmaceutical excipients comprising a sugar or a sugar alcohol.

29. The pharmaceutical composition of claim 28, wherein the potassium bumetanide has a concentration of between about 8 mg/ml and about 10 mg/mL.

30. The pharmaceutical composition of any one of claims 26-29, wherein the aqueous solution has a pH of about 6.5 to about 7.5.

31. The pharmaceutical composition of any one of claims 26-30, wherein the pharmaceutical composition comprises benzyl alcohol.

32. The pharmaceutical composition of claim 31, wherein the benzyl alcohol is in an amount of between about 0.2% (w/w) and about 1% (w/w).

33. The pharmaceutical composition of claim 32, wherein the benzyl alcohol is in an amount of about 0.5% (w/w).

34. The pharmaceutical composition of any one of claims 26-33, wherein the sugar alcohol is mannitol.

35. The pharmaceutical composition of claim 34, wherein the mannitol is in an amount of between about 2.0% (w/w) and about 5% (w/w).

36. The pharmaceutical composition of claim 35, wherein the mannitol is in an amount of about between about 2.5% (w/w) and about 4% (w/w).

37. The pharmaceutical composition of any one of claims 26-36, wherein the pharmaceutical composition comprises a buffer selected from citrate buffer and phosphate buffer.
